SEQUENCE LISTING (10 sequences)

SEQ ID NO: 1 - protein, 10 aa, synthetic construct
GYAFTNNLIE

SEQ ID NO: 2 - protein, 17 aa, synthetic construct
VINPGSGDTNYNEKFKG

SEQ ID NO: 3 - protein, 11 aa, synthetic construct
GDYYGGFAVDY

SEQ ID NO: 4 - protein, 11 aa, synthetic construct
RTSQDIGNYLN

SEQ ID NO: 5 - protein, 7 aa, synthetic construct
YTSRLHS

SEQ ID NO: 6 - protein, 9 aa, synthetic construct
QQGKSLPRT

SEQ ID NO: 7 - protein, 120 aa, synthetic construct
QVQLVQSGAEVKKPGASVKVSCKASGYAFTNNLIEWVRQAPGQGLEWIGVINPGSGDTNYNEKFKGRVTMTADKSISTAYMELSRLRSDDTAVYYCARGDYYGGFAVDYWGQGTTVTVSS

SEQ ID NO: 8 - protein, 107 aa, synthetic construct
DIQMTQSPSSLSASVGDRVTITCRTSQDIGNYLNWYQQKPGKAVKLLIYYTSRLHSGVPSRFSGSGSGTDYTLTISSLQPEDIATYFCQQGKSLPRTFGGGTKVEIK

SEQ ID NO: 9 - protein, 446 aa, synthetic construct
QVQLVQSGAEVKKPGASVKVSCKASGYAFTNNLIEWVRQAPGQGLEWIGVINPGSGDTNYNEKFKGRVTMTADKSISTAYMELSRLRSDDTAVYYCARGDYYGGFAVDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQMTQSPSSLSASVGDRVTITCRTSQDIGNYLNWYQQKPGKAVKLLIYYTSRLHSGVPSRFSGSGSGTDYTLTISSLQPEDIATYFCQQGKSLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0167717 A1
(43) Pub. Date: Jun. 18, 2026

(54) ANTI CARCINOEMBRYONIC ANTIGEN-RELATED CELL ADHESION MOLECULE 1 (CEACAM1) ANTIBODIES FOR INHIBITION OF NEUTROPHIL EXTRACELLULAR TRAPS (NET)-MEDIATED ACTIVITIES

(71) Applicant: FAMEWAVE LTD., Rehovot (IL)

(72) Inventors: Hadas REUVENI, Har Adar (IL); Tomer MEIRSON, Tel Aviv (IL); Michael SCHICKLER, Mazkeret Batya (IL)

(73) Assignee: FAMEWAVE LTD., Rehovot (IL)

(21) Appl. No.: 19/128,384

(22) PCT Filed: Nov. 9, 2023
(86) PCT No.: PCT/IL2023/051153

§ 371 Date: May 8, 2025

Related U.S. Application Data
(60) Provisional application No. 63/424,144, filed on Nov. 10, 2022.
(60) Provisional application No. 63/584,181, filed on Sep. 21, 2023.

(51) Int. Cl. C07K 16/28; A61K 39/00; A61P 7/02; G01N 33/573

(52) U.S. Cl. CPC C07K 16/2803; A61P 7/02; G01N 33/573; A61K 2039/505; G01N 2333/908; G01N 2800/226; G01N 2800/52

(57) ABSTRACT

Pharmaceutical compositions comprising anti-CEACAM1 mAbs, and their use in inhibition of NET-mediated activities, and in prevention and treatment of pathologies associated with these activities are provided and exemplified with the mAb CM24 that is also shown to effectively inhibit cancer cell migration and NET-induced platelet aggregation. Further presented is a prognostic method based on NET-biomarkers for use in patient selection and monitoring of treatment efficacy.

### FIELD OF THE INVENTION

The present invention is in the fields of immunotherapy and cell biology and relates to CEACAM1-taregeted antibodies for prevention and treatment of processes and disorders that involve activity of Neutrophil Extracellular Traps (NETs).

### BACKGROUND OF THE INVENTION

Neutrophils are the most abundant circulating leukocytes in humans and are essential components of the host response against pathogens. During infections, neutrophils migrate from peripheral blood to tissues in response to several chemotactic stimuli released within the inflammatory site. They can rapidly kill pathogens following phagocytosis, but also through the release of their potent antimicrobial arsenal, which includes granular enzymes and proteins, oxidants (Reactive Oxygen Species: ROS), as well as Neutrophil Extracellular Traps (NETs). Neutrophils also infiltrate tumors and were proposed as key mediators of neoplastic transformation, tumor progression, angiogenesis and modulation of the immune response.

Cancer-related inflammation is a driving force of tumor initiation and progression, enabling the ability of cancer cells to escape immunosurveillance. Infiltrating and resident immune cells in tumor microenvironment (TME) play a fundamental role in tumor growth, formation of metastasis and response to immunotherapies. There is increasing evidence that circulating and infiltrating neutrophils also play multiple roles in tumor initiation and progression.

NETs are extracellular decondensed chromatin networks that can include granule proteins, DNA, histones, and other matter. NETs are generated by neutrophils to engulf and kill pathogens, and may form during infection, inflammation, and/or thrombosis. More specifically, when a neutrophil detects a pathogen, granule proteins, DNA, and/or histones may combine within the neutrophil. The neutrophil can then eject the combined granule proteins, DNA, and/or histones, by disintegration of the nuclear and granular membranes permitting intracellular material to be “ejected” from the cell, to form NETs. The NETs can then capture, bind, engulf, and/or kill the pathogen in a process called NETosis. Because they originate from neutrophils and because they play a role in combating infection, NETs are commonly found extravascularly in inflamed or infected tissues.

Various markers have been tested to demonstrate NETosis. The methods to evaluate NETosis include detection of co-localized neutrophil-derived proteins and extracellular DNA and citrullinated histones, as well as detection of NET remnants in fluid samples and flow cytometric detection of cell-appendant NET components. NETs markers include: myeloperoxidase (MPO), Neutrophil Elastase (NE), Peptidyl Arginine Deiminase 4 (PAD4), Citrullinated Histone H3 (Cit-H3) and cell free DNA. Focusing on specificity, objectivity, and quantitatively of NET marker, the well accepted NET markers that were used in clinical studies are MPO/NE DNA complex (Mi-Hyun et al. J. Rheumatol. 2019 December 46 (12): 1560-1569 and Sakiko et al., Clin Chim Acta. 2016 Aug. 1:459:89-93), CitH3 together with cell free DNA (Pranav et al. Front Immunol. 2019 Jan. 24:10:28).

NETS are also involved in cancer immunoediting, progression, metastatic spread, and play a key role in the TME. NETs intensify tumor aggressiveness by enhancing cancer migration and invasion capacity and can entrap circulating cancer cells to facilitate metastasis formation and spread.

Carcinoembryonic antigen-related cell adhesion molecule 1 (CEACAM1), also known as cluster of differentiation 66a (CD66a), is a member of the carcinoembryonic antigen (CEA) gene family and belongs to the immunoglobulin (Ig) superfamily. CEACAM1 is an immune checkpoint protein that is upregulated in T and NK cells upon activation and its homophilic interactions lead to inhibition of lymphocytes' cytotoxic effect. Studies of several human tumor types have suggested that the exploitation of the CEACAM1 pathway may permit immune evasion by tumors. CEACAM1 plays an important role in tumor immune evasion, metastasis, and angiogenesis and its expression on primary cutaneous melanoma lesions strongly predicts the development of metastatic disease with poor prognosis. Moreover, increased CEACAM1 expression was observed on NK cells derived from some patients with metastatic melanoma compared with healthy donors. Preclinical animal models of tumors have shown that blockade of CEACAM1 interactions by monoclonal antibodies (mAbs) can enhance the immune response to tumors. CEACAM1 was also suggested as a putative therapeutic target to prevent metastatic progression of colon carcinoma (Rayes et al., Immunol. 2020 Apr. 15; 204 (8): 2285-2294).

CEACAM1 is associated with angiogenesis and is an inter-cellular adhesion regulator of Fas-mediated apoptosis via interaction with β-catenin that enhances natural killer cell cytotoxicity against tumor cells. High CEACAM1 expression is known to be associated with poor disease prognosis in a number of tumor types. CEACAM1 and CEACAM1-CEACAM5 pathways prevent the death of tumor cells through the inhibition of the immune activity of tumor infiltrating lymphocytes (TILs), lowering phosphorylation of immuno-receptors, and reducing SHP1/2 phosphorylation level in T and NK cells.

WO 2013/054331 discloses mAbs specific to human CEACAM1 that comprise specific sets of complementarity determining regions (CDRs). Chimeric antibodies are also disclosed, including CM10 that is a human/mouse antibody.

WO2015166484 discloses humanized anti CEACAM1 mAbs having a specific set of CDR sequences and several backmutations in the human frameworks. Among the claimed mAbs is the highly potent mAb denoted CM24.

CM24 is a humanized IgG4 mAb that binds specifically and with high affinity to the extracellular domain of CEACAM1. CM24 is a first-in-class clinical stage mAb targeting CEACAM1, with significant potential to treat multiple cancers. Blocking CEACAM1-CEACAM1 and CEACAM1-CEACAM5 interactions with CM24 is associated with anti-angiogenic, immune access, and checkpoint release mechanisms and enables cytotoxic activity of lymphocytes and the killing of tumor cells by T and NK cells.

CM24 is currently in an open-label, multicenter, multi-dose escalation, and dose expansion trial (https://clinicaltrials.gov/ct2/show/NCT04731467), in combination with Nivolumab (anti PD-1), in adults with selected advanced solid tumors (e.g., advanced recurrent refractory non-small cell lung cancer, and metastatic pancreatic cancer).

There is an unmet need to provide efficient agents that are capable of inhibiting NET-mediated activities, for prevention and treatment of a wide range of diseases and disorders associated with these activities.

### SUMMARY OF THE INVENTION

The present invention provides pharmaceutical compositions comprising the humanized anti-CEACAM1 mAb CM24, or mAbs comprising the same set of CDR sequences, and their use in inhibition of NET-mediated activities and in prevention and treatment of pathologies associated with these activities.

As NET-mediated activities are involved in a wide range of pathologies, including cardiovascular, hematological, autoimmune, neoplastic, and inflammatory diseases, anti CEACAM1 mAbs and particularly CM24 may be efficiently used for inhibiting or delaying the NET-induced processes of these pathologies.

The present invention is based on the favorable outcome of utilizing CM24 in blocking several NET-mediated activities. In a recent dose escalation part of a Phase 1/2 study of CM24 and nivolumab (NCT 04731467) and in an exploratory study that were conducted as part of this trial, it was unexpectedly found that in pancreatic cancer patients, CM24 therapy leads to a significant reduction in the levels of serum NETs and that this reduction was significant at least two weeks following treatment of CM24/Nivolumab (durable effect). It was further advantageously found that pretreatment serum levels of the NET marker myeloperoxidase (MPO) are increased in patients that showed stable disease (SD) or partial response (PR) and survived longer and not in patients who showed no response at all (progressive disease, PD), suggesting serum MPO as a biomarker for patient selection for CM24 therapy. In part C2 of the study, a significant and durable decrease in serum MPO was detected in patients who showed SD or PR and not in those that haven't responded at all (PD). It is therefore disclosed for the first time that MPO may be used as a prognostic biomarker for selecting patients eligible for treatment with anti-CEACAM1 antibodies, and for monitoring the effectiveness of treatment with such antibodies.

In cancer, blocking, inhibiting, or delaying these NET-mediated activities will result in blocking, inhibiting, or delaying intravasation into the vasculature, obstructing the cancer cells' survival within the bloodstream, extravasation into the organ parenchyma and formation of dormant cells or multicellular micrometastases and macrometastases.

It is herein shown for the first time, that CM24 effectively inhibits cancer cell migration in-vivo, and suppresses metastatic activity, thereby curtailing the progression of the disease. The present invention provides a method of utilizing CM24, mAbs comprising the same set of CDR sequences, or antibody fragments comprising at least the binding site of CM24, to block the metastatic cascade facilitated by NETosis, thus effectively obstructing the metastatic cascade and preventing seeding, spreading and subsequent exponential growth of distant metastatic colonies. The present invention is advantageous, as utilizing immunological methods for treatment and even prevention in some cases, of pathological conditions such as cancer and NET-associated thrombotic diseases and disorders, rather than relying on cytotoxic treatments, greatly reducing the deleterious side effects associated with such treatments.

The present invention further provides a method of selecting a subject diagnosed with cancer, for treating with an anti-CEACAM1 mAb, or a fragment or conjugate thereof, or for monitoring treatment effectiveness, wherein the level of a NET-marker, e.g., myeloperoxidase (MPO) is used to optimize patient selection and monitor treatment efficacy.

The present invention thus provides according to one aspect, a method of preventing or inhibiting a NET-mediated activity, comprising utilizing a mAb, or an active fragment or conjugate thereof, comprising a set of six CDR sequences wherein heavy chain CDR1 (HC-CDR1) comprises the sequence GYAFTNNLIE (SEQ ID NO: 1), heavy chain CDR2 (HC-CDR2) comprises the sequence VINPGSGDTNYNEKFKG (SEQ ID NO: 2), heavy chain CDR3 (HC-CDR3) comprises the sequence GDYYGGFAVDY (SEQ ID NO: 3), light chain CDR1 (LC-CDR1) comprises the sequence RTSQDIGNYLN (SEQ ID NO: 4), light chain CDR2 (LC-CDR2) comprises the sequence YTSRLHS (SEQ ID NO: 5), and light chain CDR3 (LC-CDR3) comprises the sequence QQGKSLPRT (SEQ ID NO: 6).

According to some embodiments, the mAb or fragment thereof comprises a set of six CDR sequences wherein, HC-CDR1 consists of GYAFTNNLIE (SEQ ID NO: 1), HC-CDR2 consists of VINPGSGDTNYNEKFKG (SEQ ID NO: 2), HC-CDR3 consists of GDYYGGFAVDY (SEQ ID NO: 3), LC-CDR1 consists of RTSQDIGNYLN (SEQ ID NO: 4), LC-CDR2 YTSRLHS consists of (SEQ ID NO: 5), and LC-CDR3 consists of QQGKSLPRT (SEQ ID NO: 6).

The present invention also provides according to another aspect, a method of preventing, inhibiting, or delaying a pathological process or condition that involves a NET-mediated activity, comprising administering to a subject in need thereof, a mAb, or an active fragment thereof, comprising a set of six CDR sequences wherein, HC-CDR1 consists of GYAFTNNLIE (SEQ ID NO: 1), HC-CDR2 consists of VINPGSGDTNYNEKFKG (SEQ ID NO: 2), HC-CDR3 consists of GDYYGGFAVDY (SEQ ID NO: 3), LC-CDR1 consists of RTSQDIGNYLN (SEQ ID NO: 4), LC-CDR2 YTSRLHS consists of (SEQ ID NO: 5), and LC-CDR3 consists of QQGKSLPRT (SEQ ID NO: 6).

The present invention also provides, according to another aspect, a pharmaceutical composition comprising a mAb, or an active fragment thereof, comprising a set of six CDR sequences wherein HC-CDR1 consists of GYAFTNNLIE (SEQ ID NO: 1), HC-CDR2 consists of VINPGSGDTNYNEKFKG (SEQ ID NO: 2), HC-CDR3 consists of GDYYGGFAVDY (SEQ ID NO: 3), LC-CDR1 consists of RTSQDIGNYLN (SEQ ID NO: 4), LC-CDR2 YTSRLHS consists of (SEQ ID NO: 5), and LC-CDR3 consists of QQGKSLPRT (SEQ ID NO: 6), and a pharmaceutically acceptable salt, carrier or diluent, for use in the prevention or delaying of NET-mediated activities and for preventing, inhibiting, or delaying a pathological process or disorder that involves a NET-mediated activity.

In some embodiments, the anti-CAECAM1 antibody is a chimeric antibody. In other embodiments, the anti CEACAM1 antibody is a humanized antibody or a partially humanized antibody.

In other embodiments, anti CEACAM1 antibody comprises a heavy chain variable region comprising the sequence QVQLVQSGAEVKKPGASVKVSCKASGYAFTNNLIEWVRQAPGQGLEWIGVINPGSG DTNYNEKFKGRVTMTADKSISTAYMELSRLRSDDTAVYYCARGDYYGGFAVDYWG QGTTVTVSS (SEQ ID NO: 7), and a light chain variable region comprising the sequence

(SEQ ID NO: 8)
DIQMTQSPSSLSASVGDRVTITCRTSQDIGNYLNWYQQKPGKAVKLLIY
YTSRLHSGVPSRFSGSGSGTDYTLTISSLQPEDIATYFCQQGKSLPRTF
GGGTKVEIK,

or an active fragment thereof comprising at least the binding site, or an antibody analog or derivative thereof having at least 90% identity with any of said chain sequences.

According to some embodiments, the heavy chain variable region of the antibody comprises an amino acid sequence at least about 95% identical SEQ ID NO: 7, and the light chain variable region comprises an amino acid sequence at least about 95% identical to SEQ ID NO: 8. According to some embodiments, the heavy chain variable region of the antibody comprises an amino acid sequence at least about 97% identical SEQ ID NO: 7, and the light chain variable region comprises an amino acid sequence at least about 97% identical to SEQ ID NO: 8. According to some embodiments, the heavy chain variable region of the antibody comprises an amino acid sequence at least about 99% identical SEQ ID NO: 7, and the light chain variable region comprises an amino acid sequence at least about 99% identical to SEQ ID NO: 8. Each option represents a separate embodiment of the present invention.

According to some embodiments, the antibody or fragment thereof is an IgG mAb. According to some embodiments, the anti CEACAM1 mAb has a heavy chain constant region selected from IgG4, IgG1, and IgG2. In other embodiments, the antibody comprises a human IgG constant region selected from IgG1 and IgG4. In certain embodiments, the humanized antibody or fragment thereof is an IgG4 subclass. In certain embodiments, the humanized antibody or antigen binding fragment thereof is an IgG1 subclass. In some embodiments, the anti CEACAM1 antibody comprises a human kappa light chain constant region. Each option represents a separate embodiment of the present invention.

In exemplary embodiments, the anti CEACAM1 antibody is CM24, comprising a heavy chain sequence QVQLVQSGAEVKKPGASVKVSCKASGYAFTNNLIEWVRQAPGQGLEWIGVINPGSG DTNYNEKFKGRVTMTADKSISTAYMELSRLRSDDTAVYYCARGDYYGGFAVDYWG QGTTVTVSSASTKGPSVFPLAPCSRSTSESTAALGCLVKDYFPEPVTVSWNSGALTSG VHTFPAVLQSSGLYSLSSVVTVPSSSLGTKTYTCNVDHKPSNTKVDKRVESKYGPPCP PCPAPEFLGGPSVFLFPPKPKDTLMISRTPEVTCVVVDVSQEDPEVQFNWYVDVEVH NAKTKPREEQFNSTYRVVSVLTVLHQDWLNGKEYKCKVSNKGLPSSIEKTISKAKGQ PREPQVYTLPPSQEEMTKNQVSLTCLVKGFYPSDIA VEWESNGQPENNYKTTPPVLDS DGSFFLYSRLTVDKSRWQEGNVFSCSVMHEALHNHYTQKSLSLSLGK (SEQ ID NO: 9), and the light chain sequence DIQMTQSPSSLSASVGDRVTITCRTSQDIGNYLNWYQQKPGKAVKLLIYYTSRLHSGV PSRFSGSGSGTDYTLTISSLQPEDIATYFCQQGKSLPRTFGGGTKVEIKRTVAAPSVFIF PPSDEQLKSGTASVVCLLNNFYPREAKVQWKVDNALQSGNSQESVTEQDSKDSTYSL SSTLTLSKADYEKHKVYACEVTHQGLSSPVTKSFNRGEC (SEQ ID NO: 10), or an active fragment thereof comprising at least the binding site, or an antibody analog or derivative thereof having at least 90% identity with any of said chain sequences.

The antibodies of the methods and compositions of the present invention also include conjugates comprising the antibodies or fragments thereof. A conjugate may comprise, according to some embodiments, an antibody or a fragment thereof attached to a cytotoxic moiety, a radioactive moiety, or an affinity or labeling tag.

Any disease or disorder in which NETs are highly involved in its pathogenesis or progression and/or in which inhibition of NETs activity will result in improvement, delay of symptoms etc., is eligible for treatment with the mAbs of the present invention.

In some embodiments, the subject in need of a treatment has been diagnosed with a neoplastic disease, namely with cancer. In some embodiments, the neoplastic disease is a solid tumor. According to some embodiments, the cancer is a metastatic cancer or tumor. In some embodiments, the neoplastic disease is selected from the group consisting of carcinoma, lymphoma, blastoma, sarcoma, melanoma, unknown primary, skin, lung, thyroid, parathyroid, breast, cardiac, thymic, bone, soft-tissue, brain, retinal, ophthalmologic, head and neck, esophageal, gastric, colorectal, prostate, pancreatic, biliary, hepatic, bladder, adrenal, renal, genito-urinal, testicular, cervical, fallopian, ovarian, uterine, vulvar, or endometrial cancer. In some embodiments, the neoplastic disease is a hematological cancer. In some embodiments, the hematological cancer is selected from lymphoma, leukemia, myelodysplastic syndromes, myeloproliferative disorders and myeloma. Each option represents a separate embodiment of the present invention.

According to some embodiments the cancer is selected from the group consisting of pancreatic cancer, lung cancer and melanoma.

According to some embodiments, treatment with the anti CEACAM1 antibody or antibody fragments of the present invention result in prevention, inhibition, or delay of at least one of: formation of metastases, migration or spread of metastases, adhesion of metastases, intravasation of cancerous cells into the vasculature, cancer cells' survival within the bloodstream, extravasation of cancer cells into the organ parenchyma, and formation of dormant cells or multicellular metastases.

In an additional aspect, the present invention provides a method of preventing, delaying, or inhibiting the formation, migration, spread, adhesion, or progression of metastases, comprising administering the mAb to CEACAM1 defined above.

In some embodiments, formation, migration or spread of metastases following tumor resection surgery is prevented or inhibited.

In some embodiments, the patient undergoing the surgery has been treated with additional anti-cancer therapies, selected from the group consisting of chemotherapies, radiation, and immunotherapies.

The present invention further provides an anti-CEACAM1 mAb for use in treating a NET-mediated disorder or complication in a subject in need of such treatment, the method comprises:
(i) determining the level of at least one NET-biomarker in a biological sample obtained from a subject;
(ii) comparing the at least one NET-biomarker level to a reference value of a control sample value; and
(iii) administering anti-CEACAM1 antibody to said subject if said NET-biomarker level in the sample is significantly higher than the reference value or the control sample value.

According to some embodiments, the subject is a patient diagnosed with cancer or suspected to have cancer.

According to some embodiments, the subject is diagnosed with or is suspected to have a non-malignant NET-related disease, disorder, or complication, including treatment-induced complication.

According to some embodiments, the levels of at least two NET-biomarkers are measured.

Any marker known in the art to detect NETosis may be used with the methods of the present invention. According to some embodiments, detection NETosis comprises detection of co-localized neutrophil-derived proteins and extracellular DNA and citrullinated histones, detection of NET remnants in fluid samples, and flow cytometric detection of cell-appendant NET components. NETs markers include but are not limited to: myeloperoxidase (MPO), Neutrophil Elastase (NE), Peptidyl Arginine Deiminase 4 (PAD4), Citrullinated Histone H3 (Cit-H3) and cell free DNA. According to some embodiments, the at least one marker is selected from MPO, NE and DNA complex.

According to some embodiments, the at least one NET marker is selected from the group consisting of myeloperoxidase (MPO), Neutrophil Elastase (NE), Peptidyl Arginine Deiminase 4 (PAD4), Citrullinated Histone H3 (Cit-H3) and cell free DNA. According to some embodiments, the at least one NET marker is selected from MPO, NE and DNA complex. According to specific embodiments, the NET marker is MPO. Each option represents a separate embodiment of the present invention.

According to some specific embodiments, the NET-biomarker is MPO,

The present invention thus provides, according to some embodiments, an anti-CEACAM1 mAb for use in treating cancer in a subject in need of such treatment, the method comprises:
(i) determining the level of myeloperoxidase (MPO) in a biological sample obtained from a subject diagnosed with cancer;
(ii) comparing the MPO level to a reference value of a control sample value; and
(iii) administering anti-CEACAM1 antibody to said subject if the MPO level in the sample is significantly higher than the reference value or the control sample value.

In some embodiments, the significantly higher MPO level is equivalent to at least about 100%, at least about 200%, or at least about 300% increase relative to the reference value or the control sample value.

In another aspect, the present invention provides a method of treating cancer in a subject in need of such treatment, comprising:
(i) determining the level of MPO in a biological sample obtained from a subject diagnosed with cancer;
(ii) comparing the MPO level to a reference value or a control sample value; and
(iii) administering anti CEACAM1 to the subject if the MPO level in the sample is significantly higher than the reference value or the control sample value.

In some embodiments, the biological sample is a blood sample. In still other embodiments, the blood sample is selected from whole blood, serum and plasma. In other embodiments, the biological sample obtained from the subject is a biopsy, e.g., a tissue or a liquid biopsy or particularly a tumor biopsy.

The present invention further provides a method of selecting a subject amenable to an anti-CEACAM1 antibody treatment, comprising the steps of: (i) providing a biological sample from the subject; (ii) determining the level of at least one NET-biomarker in the sample of step (i), and (iii) comparing the at least one NET-biomarker level to a reference value or to a control sample value, wherein a significant increase in the level of said NET-biomarker relative to the reference value or the control sample value indicates that the subject is likely to respond therapeutically to the anti-CEACAM1 antibody.

According to some embodiments, the subject is diagnosed with cancer or is suspected to have a cancer.

According to some embodiments, the subject is diagnosed with or is suspected to have a non-malignant NET-related disease, disorder, or complication, including treatment-induced complication.

According to some embodiments, the at least one NET marker is selected from the group consisting of myeloperoxidase (MPO), Neutrophil Elastase (NE), Peptidyl Arginine Deiminase 4 (PAD4), Citrullinated Histone H3 (Cit-H3) and cell free DNA. According to some embodiments, the at least one NET marker is selected from MPO, NE and DNA complex. According to specific embodiments, the NET marker is MPO. Each option represents a separate embodiment of the present invention.

According to some embodiments, the levels of at least two NET markers are measured.

According to some specific embodiments, the NET marker is MPO and the invention provides a method of selecting a cancer subject amenable to an anti-CEACAM1 antibody treatment, comprising the steps of: (i) providing a biological sample from the subject; (ii) determining the level of MPO in the sample of step (i), and (iii) comparing the MPO level to a reference value or to a control sample value, wherein a significant increase in the level of MPO relative to the reference value or the control sample value indicates that the subject is likely to respond therapeutically to the anti-CEACAM1 antibody.

In some embodiment the increase in MPO levels relative to the reference value or the control sample value is equivalent to an increase of at least about 100%. In other embodiments, the increase is equivalent to at least about 200%. In still other embodiments, the increase is equivalent to at least about 300%. In still more embodiments, an increase in the level of MPO identified in the patient, characterizes said patient as being expected to develop a severe form of the cancer.

In some embodiments, the anti-CEACAM1 mAb or antibody fragment comprises a set of CDR sequences consisting of SEQ ID Nos. 1-6. In other embodiments, the anti-CEACAM1 mAb is CM24.

In some embodiments, the subject selected for treatment has been diagnosed with a solid tumor cancer. In other embodiments, the solid tumor cancer is selected from pancreatic, lung and melanoma cancers. In other embodiments, the patient received the anti-CEACAM1 mAb therapy in combination with at least one other anti-cancer treatment, e.g., chemotherapy. In specific embodiments, the patient received the anti-CEACAM1 mAb therapy in combination with an anti-PD-1 antibody therapy.

In another aspect, a method of inhibition of a treatment-induced thrombosis is provided, comprising administering an anti CEACAM1 mAb or a fragment thereof comprising a set of six CDR sequences wherein, heavy chain CDR1 (HC-CDR1) comprises the sequence GYAFTNNLIE (SEQ ID NO: 1), heavy chain CDR2 (HC-CDR2) comprises the sequence VINPGSGDTNYNEKFKG (SEQ ID NO: 2), heavy chain CDR3 (HC-CDR3) comprises the sequence GDYYGGFAVDY (SEQ ID NO: 3), light chain CDR1 (LC-CDR1) comprises the sequence RTSQDIGNYLN (SEQ ID NO: 4), light chain CDR2 (LC-CDR2) comprises the sequence YTSRLHS (SEQ ID NO: 5), and light chain CDR3 (LC-CDR3) comprises the sequence QQGKSLPRT (SEQ ID NO: 6). According to some embodiments, the mAb is CM24. According to some embodiments, the treatment that induces thrombosis is selected from immunotherapy, surgery, radiation, hormone-therapy and chemotherapy.

In an additional aspect there is provided a method for administering an anti-CEACAM1 antibody for the inhibition of cancer cell invasiveness into the vasculature.

In an additional aspect there is provided a method for administering anti-CEACAM1 antibody treatment for the inhibition of cancer cell invasiveness into the surrounding extra cellular matrix.

In an additional aspect there is provided a method for administering anti-CEACAM1 antibody treatment for the inhibition of cancer cell extravasation into organ parenchyma.

In an additional aspect there is provided a method for administering anti-CEACAM1 antibody treatment for the prevention of seeding and exponential growth of distant metastatic colonies.

Any administration route suitable for delivery of proteins or antibodies may be used with the compositions and methods of the present invention and the compositions administered are formulated according to the administration mode. According to some embodiments, the mAb is administered parenterally. According to some embodiments, the mAb is administered via a route selected from intravenously, intramuscularly, subcutaneously, intra-tumorally, intra-dermally, intra-arterially, intraarticulary, intralesionally or submucosally, intranasally, orally, and topically.

Ordinarily, intravenous (i.v.), administration, by infusion or injection is used. In other embodiments, the anti-CEACAM1 composition is administered via an intra-tumoral route. In other embodiments, the composition is administered during or following surgery.

A method according to the present invention, of treating cancer or a non-cancerous NET-associated disease or disorder, comprises according to some embodiments, administering to a subject in need thereof at least one dose of a mAb to CEACAM1 described above, ranging from 0.01 mg/kg to 50 mg/kg body weight.

According to some embodiments, the at least one dose is selected from the group consisting of: 0.01-0.1 mg/kg; 0.1-1 mg/kg; 1-10 mg/kg; and 10-50 mg/kg.

According to some embodiments, the method comprises administering of multiple doses of mAb, wherein the multiple doses are identical or different. According to some embodiments, the method comprises administering multiple escalating doses. According to some embodiments, the method comprises at least one cycle of administration for at least 12 weeks.

According to some embodiments, the treatment duration is 2-60 weeks. According to other embodiments, the treatment duration is 12-50 weeks. According to some specific embodiments the treatment duration is selected from the group consisting of: 12-20 weeks, 20-30 weeks and 30-50 weeks. According to yet other embodiments, the treatment regimen comprises several administration cycles each for at least 12 weeks.

According to some embodiments, the treatment regimen comprises 1-8 cycles, each cycle comprises 2-6 infusions of the anti CEACAM mAb for a duration of at least 4 weeks. According to some embodiments the treatment regimen comprises 2-6 cycles each cycle comprises 4 infusions of the anti CEACAM mAb for a duration of at least 4 weeks.

According to some embodiments, administration is once every week, one every 2 weeks, once every 3 weeks, once every 4 weeks, or once every 5 weeks. Each possibility represents a separate embodiment of the present invention.

According to some embodiments, a treatment regimen comprises 1-10 cycles, each cycle comprising 2-5 infusions every 1-4 weeks, with a mAb described above, followed by 2-8 weeks between each cycle.

According to some embodiments a dose escalation regimen is provided comprising administration starting with 0.01 mg/kg, and continuing to 0.03 mg/kg, 0.1 mg/kg, 0.3 mg/kg, 1 mg/kg, 3 mg/kg, and 10 mg/kg. According to yet other embodiments, the treatment regimen comprises 6 cycles of 4 infusions each administered every 2 weeks.

According to some embodiments, the subject is human.

According to some embodiments, the human subject is diagnosed with a NET-associated condition. According to some embodiments, the human subject is diagnosed with cancer. According to some embodiments, the human subject is a cancer patient undergoing a tumor resection surgery.

The methods and uses provided according to the present invention, may comprise stand-alone treatments with the anti CEACAM1 antibody or fragment or as part of a treatment regimen comprising at least one additional treatment.

In some embodiments, administration of an anti CEACAM1 antibody for treatment of cancer or prevention of cancer metastases, comprises at least one additional anti-cancer therapy. According to some embodiments, the at least one additional anti-cancer therapy is selected from the group consisting of chemotherapy, radiation, surgery, and immunotherapy.

According to some embodiments, the method of treating cancer comprises administration of the anti CEACAM1 antibody or fragment described herein and an additional anti-cancer agent. According to some embodiments, the additional anti-cancer agent is selected from the group consisting of: immune-modulator, activated lymphocyte cell, immune cell therapeutic agent, kinase inhibitor and chemotherapeutic agent.

According to some embodiments, the additional immune-modulator is an inhibitor of an immune checkpoint molecule.

According to some embodiments, the immune checkpoint inhibitor inhibits the interaction between PD-1 and its ligand, PD-L1. According to some embodiments, the inhibitor of an immune checkpoint molecule is an anti PD-1 inhibitor, e.g., antibody. In other embodiments, the immune checkpoint inhibitor is an anti PD-L1 inhibitor, e.g., antibody.

In another aspect, the present invention provides a method for inhibiting the formation of NETs in a subject, comprising administering to a patient an effective dose of anti-CEACAM1 antibody, in particular CM24. In some embodiments, inhibiting the formation of NETs comprise preventing the formation of a NET and/or reducing the likelihood that a NET will form in a subject. In some embodiments, inhibiting the formation of NETs comprise inhibiting the growth or progression of pre-existing NETs and/or reducing the likelihood that a pre-existing NET will grow or progress in a subject. In some embodiments, the method of inhibiting the formation of NETs results in reducing the severity of symptoms associated with the development of NETs. In some embodiments, the symptom associates with the development of NETs is thrombosis. In some embodiments, a subject receiving treatment to inhibit the formation of NETs is a subject having or diagnosed as having a cardiovascular condition. In some embodiments, a subject receiving treatment to inhibit NET-mediated activity is a subject having or diagnosed as having a condition which makes the subject predisposed to thrombosis (i.e., prothrombotic).

According to some embodiments, the pathological condition, namely the process or disorder that involves a NET-mediated activity is a non-cancerous process or disorder. According to some embodiments, the NET-associated disease or disorder is a non-malignant thrombotic disease or disorder.

According to some embodiments of the present invention, the anti CEACAM1 mAb or antibody fragment inhibits adhesion of non-cancerous cells to NET components.

A non-cancerous conditions, diseases and disorders that involves a NET-mediated activity include but are not limited to thrombotic diseases, thrombosis, pro-thrombosis condition, venous thromboembolism, arterial thromboembolism, thromboinflammatory condition, hematological condition, cardiovascular condition, autoimmune disease, autoinflammatory disease or disorder, immune-mediated disease, systemic inflammatory condition. These and other NETs-associated conditions are eligible to treatment with the compositions and method of the present invention.

The compositions and methods of the present invention can also be referred to as thromboprophylactic. In some embodiments, the effect achieved by the compositions and methods of the present invention are that of primary thromboprophylaxis, i.e—aimed at directly minimizing the occurrence of thromboembolism, and in other embodiments that effect is that of secondary thromboprophylaxis—prevention of recurrence of thromboembolism in subjects with a history of thrombosis-related incidents.

In some embodiments, NET-associated disease or disorder is a thrombotic cardiovascular disease.

In some embodiments, the thrombotic cardiovascular disease is myocardial infarction. In some embodiments, the myocardial infarction is characterized by an abundance of NETS in the coronary thrombi of the subject. In additional embodiments, the myocardial infarction is characterized by a presence of NETS in the subject's coronary stent thrombus. In still more embodiments, the myocardial infarction is characterized by platelet-neutrophil interactions mediated through polyp, which in turn triggers NET generation.

In yet additional embodiments, the thrombotic cardiovascular disease is carotid atherosclerosis. In some embodiments, the carotid atherosclerosis is characterized by an elevation of myeloperoxidase (MPO), cell-free DNA, and MPO-DNA complexes, which are detected in intraplaque hemorrhagic segments of the carotid atherosclerosis.

In an additional embodiment, the thrombotic cardiovascular disease is cerebrovascular stroke. In some embodiments, the cerebrovascular stroke is characterized by a positive correlation between NETotic markers and clot stability as well as resistance to endovascular therapy.

In an additional embodiment, the thrombotic cardiovascular disease is deep vein thrombosis (DVT), portal vein thrombosis, or marantic endocarditis. In some embodiments, the DVT is characterized by activated neutrophils and plasma nucleosomes/DNA.

In an additional embodiment, the thrombotic cardiovascular disease is pulmonary embolism. In some embodiments, the pulmonary embolism is characterized by a NET-mediated thrombus organization and maturation.

In an additional embodiment, the thrombotic cardiovascular disease is chronic thromboembolic pulmonary hypertension. In some embodiments, the chronic thromboembolic pulmonary hypertension is characterized by a presence of NETs in the plasma and intrapulmonary thrombi of patients.

In yet other embodiments, the NET-associated condition is a hematological disease or disorder.

In some embodiments, the hematological disease is thrombotic thrombocytopeni purpura (TTP). In some embodiments, the TTP is characterized by impaired DNase1-mediated degradation of NETs.

In an additional embodiment, the hematological disease is heparin-induced thrombocytopenia or thrombosis. In some embodiments, the heparin-induced thrombocytopenia or thrombosis is characterized by neutrophil activation which leads to NETs-induced thrombosis.

In an additional embodiment, the NET-associated disease is an autoimmune disease.

In some embodiments, the autoimmune disease is systemic lupus erythematosus (SLE). In some embodiments, the SLE is characterized by a display of excessive cell death by neutrophils, resulting in NET formation.

In an additional embodiment, the autoimmune disease is antiphospholipid syndrome (APS). In some embodiments, the APS is characterized by an antiphospholipid antibody-mediated induction of NET formation.

In an additional embodiment, the autoimmune disease is rheumatoid arthritis (RA). In some embodiments, the rheumatoid arthritis is characterized by an increase of NET formation in peripheral blood and synovium.

In an additional embodiment, the autoimmune disease is psoriasis. In some embodiments, the psoriasis is characterized by a correlation between the number of NETotic cells and the severity of the disease.

In an additional embodiment, the autoimmune disease is ulcerative colitis. In some embodiments, the ulcerative colitis is characterized by a NET-mediated enhancement of procoagulant activity.

In an additional embodiment, the autoimmune disease is gout. In some embodiments, the gout is characterized by monosodium urate (MSU) crystal-induced stimulation of neutrophils to produce NETs and IL-1ß.

In an additional embodiment, the autoimmune disease is systemic sclerosis.

In an additional embodiment, the autoimmune disease is ANCA-associated vasculitis. In some embodiments, the ANCA-associated vasculitis is characterized by NET formation which triggers vasculitis and promotes the autoimmune response against neutrophil components.

In an additional embodiment, the autoimmune disease is dermatomyositis. In some embodiments, the dermatomyositis is characterized by an increase of NETs.

In an additional embodiment, the autoimmune disease is polymyositis. In some embodiments, the polymyositis is characterized by an increase of NETs.

In an additional embodiment, the NET-associated condition is a systemic inflammatory response syndrome.

In some embodiments, the systemic inflammatory response syndrome is sepsis or septic shock. In other embodiments, the systemic inflammatory response syndrome is caused by viral infection. According to some embodiments, the viral infection is of SARS-CoV-2.

In an additional embodiment, the systemic inflammatory response syndrome manifests as disseminated intravascular coagulation (DIC).

In another aspect, a method of inhibiting the formation of NETs in a subject is provided, the method comprising administering to a patient an effective dose of an anti-CEACAM1 antibody.

In another aspect, the present invention provides a kit for selecting subjects amenable to an anti-CEACAM1 antibody treatment or for predicting the response of a subject to an anti-CEACAM1 antibody, the kit comprises means for determining the level of at least one NET-biomarker in a biological sample, means for comparing the expression level of the at least one NET-biomarker to a reference value or to a control sample value; and instruction material directing the correlation between the ratio of said NET-biomarker to the reference level. In some embodiments, the subjects are diagnosed with cancer. In other embodiments, the subjects are diagnosed with non-malignant NET-related disease, disorder, or complication. In some embodiments, the subjects are diagnosed with thrombotic-associated conditions. In still more embodiments, the subjects are diagnosed with an autoimmune disease or with a rheumatologic disorder.

In some embodiments, the at least one NET marker is selected from the group consisting of myeloperoxidase (MPO), Neutrophil Elastase (NE), Peptidyl Arginine Deiminase 4 (PAD4), Citrullinated Histone H3 (Cit-H3) and cell free DNA. According to some embodiments, the at least one NET marker is selected from MPO, NE and DNA complex. According to specific embodiments, the NET marker is MPO. Each option represents a separate embodiment of the present invention.

In some embodiments, the NET-biomarker is MPO, and the invention provides a kit for selecting subjects amenable to an anti-CEACAM1 antibody treatment or for predicting the response of a subject to an anti-CEACAM1 antibody, the kit comprises means for determining the level of MPO in a biological sample, means for comparing the expression level of the MPO to a reference value or to a control sample value; and instruction material directing the correlation between the ratio of the MPO to the reference level. In some embodiments, the subjects are diagnosed with cancer. In other embodiments, the subjects are diagnosed with non-malignant NET-related disease, disorder, or complication. In other embodiments, the subjects are diagnosed with thrombotic-associated conditions. In still more embodiments, the subjects are diagnosed with an autoimmune disease or with a rheumatologic disorder.

In some embodiments, the subject is a cancer patient or the subject is suspected to have cancer and thus, the invention provides a kit for selecting cancer subject amenable to an anti-CEACAM1 antibody treatment or for predicting the response of a cancer subject to an anti-CEACAM1 antibody, the kit comprises means for determining the level of MPO in a biological sample, means for comparing the expression level of the MPO to a reference value or to a control sample value; and instruction material directing the correlation between the ratio of the MPO to the reference level.

According to some embodiments, of any of the aspects of the present invention, the anti CEACAM1 mAb or fragment thereof comprises a set of six CDR sequences wherein, HC-CDR1 comprises SEQ ID NO: 1, HC-CDR2 comprises SEQ ID NO: 2, HC-CDR3 comprises SEQ ID NO: 3, LC-CDR1 comprises SEQ ID NO: 4, LC-CDR2 comprises SEQ ID NO: 5, and LC-CDR3 comprises SEQ ID NO: 6.

According to some embodiments, of any of the aspects of the present invention, the anti CEACAM1 mAb or fragment thereof comprises a set of six CDR sequences wherein, HC-CDR1 consists of SEQ ID NO: 1, HC-CDR2 consists of SEQ ID NO: 2, HC-CDR3 consists of SEQ ID NO: 3, LC-CDR1 consists of SEQ ID NO: 4, LC-CDR2 consists of SEQ ID NO: 5, and LC-CDR3 consists of SEQ ID NO: 6.

According to some embodiments, of any of the aspects of the present invention, the anti CEACAM1 mAb or fragment thereof comprises a heavy chain variable region of SEQ ID NO: 7 or a variant having at least 90% identity, and a light chain variable region of SEQ ID NO: 8, or a variant having at least 90% identity.

In exemplary embodiments, of any aspect of the present invention, the anti CEACAM1 antibody is CM24, comprising a heavy chain sequence set forth in SEQ ID NO: 9, and the light chain sequence set forth in SEQ ID NO: 10, or an active fragment thereof comprising at least the binding site, or an antibody analog or derivative thereof having at least 90% identity with any of said chain sequences.

According to some embodiments, of any of the aspects of the present invention, the antibody or fragment thereof is an IgG mAb comprising a heavy chain constant region selected from IgG4, IgG1, and IgG2. In other embodiments, the antibody comprises a human IgG constant region selected from IgG1 and IgG4. In some embodiments, the anti CEACAM1 antibody comprises a human kappa light chain constant region. Each option represents a separate embodiment of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1C. Illustrates CM24-mediated suppression of NET-promoting cancer cell migration in vitro. The following human cancer cell lines were treated with CM24, isotype control, or left untreated in a serum-free medium with or without NETs: melanoma SK-MEL-28 (FIG. 1A), non-small cell lung carcinoma (NSCLC) A549 (FIG. 1B), and pancreatic cancer AsPC1 (FIG. 1C). Serum was used as a chemotactic stimulus for cancer cell migration in a transwell Boyden chamber. Fluorescence of the migrating cells was measured every 2 hr for 24 hr (SKMEL-28, A549) or 48 hr (AsPC1) and the mean AUC±SEM is presented.

Differences between the non-treated (No treat) or isotype controls and the CM24-treated wells were calculated by two-way ANOVA statistical analysis. Significancy is indicated by *p<0.05, ****p<0.0001, and ns for not significant.

FIGS. 2A-2F depicts confocal microscopy images of NETs demonstrating co-localization of CEACAM1 and NETs and direct CM24 binding to NET, as evident by areas of CM24/MPO/extracellular DNA overlap. DAPI staining is used to visualize the extracellular DNA component of NETs (FIGS. 2A-2D, 2F), anti-MPO antibody for NET marker (FIGS. 2A-2C), and CEACAM1 on the NET structure is stained by CM24 (FIGURES A,C, and E emphasized by white arrows). Zoomed-in pictures are demonstrated in FIGS. 2C-2E.

FIG. 3 depicts the serum MPO levels (ng/ml) measured in 30 healthy donors and 10 pancreatic ductal adenocarcinoma (PDAC) patients before treatment (P<0.01).

FIG. 4A-4B illustrate the percent reduction in serum MPO levels in patients following treatment with CM24 and Nivolumab. MPO levels were analyzed in serum samples of the patients in Part A of the clinical study, including ten PDAC patients and two colorectal (CRC) patients (FIG. 4A) or ten PDAC patients (FIG. 4B). Measurements by ELISA were performed at five time points: C1D1 (Cycle 1 Day 1) pre-dose; end on CM24 infusion (EOI); 1.5 hours following EOI; C1D15 (Cycle 1 day 15) pre-dose; and C1D15 EOI.

FIGS. 5A-5B depict MPO levels in serum samples of PDAC patients before treatment with the combination of CM24, Nivolumab and the chemotherapy cocktail Nal-Irinotecan/5FU/LV. FIG. 5A illustrates MPO levels, the time know alive and the best response (PD=progressed disease, PR=partial response, SD=stable disease) per patient. FIG. 5B compares the average serum MPO levels in patients who showed disease control (SD, PR) versus patients who progressed on treatment (P<0.05). Mean MPO levels in healthy controls (N=30) is depicted by the dotted lines.

FIGS. 6A-6B depict a significant reduction in serum MPO, two weeks following treatment with a combination of CM24, Nivolumab and Nal-Irinotecan/5FU/LV, in patients who exhibited disease control (PR or SD) as opposed to patients who progressed (PD). FIG. 6A illustrates the percentage of MPO relative to pre-dose for each patient and FIG. 6B illustrates the average values in patients who exhibited disease control versus those who progressed (P=0.0001).

FIG. 7 depicts measurement of MPO levels per concentration (ng/uL) of NETs. MPO levels were measured in various quantities of fresh or frozen NETs (5, 10 and 20 ng/uL) by ELISA. MPO levels are presented as average of quadrants.

FIG. 8 depicts the dose dependent NET-induced platelet aggregation. Platelets were combined with 5, 10, and 20 ng/μl NETs and aggregation was quantified. Bars are means with N=4, +/−SEM. Two-way ANOVA was used to determine significance (***p<0.001, ****p<0.0001).

FIG. 9 depicts the CM24-induced inhibition of adenosine diphosphate (ADP)-induced platelet aggregation. Platelet reach plasma was incubated with CM24 or hIgG4 (as isotype control) for 30 minutes, with or without ADP as an inducer of platelet aggregation, and platelet aggregation was measured. Two-way ANOVA was used for selected comparisons (****p<0.0001).

FIG. 10 depicts the inhibition of NET-induced platelet aggregation by CM24. Platelet rich plasma was pre-treated with CM24 or isotype control antibodies and added to fresh NETs at 5 and 10 ng/uL concentrations. Graph presents mean AUC+/−SEM (N=4) of aggregation over 30 min of assay. Two-way ANOVA with multiple comparisons was performed to determine statistical difference between treatments (****p<0.0001).

FIG. 11 depicts the interference with adhesion of CEACAM1 expressing melanoma cells to NETs by CM24. FACS analysis of CEACAM1 positive SK-MEL-28 cells vs. CEACAM1 negative Jurkat cells was performed following incubation with CM24 or isotype control for 30 minutes. The graph presents NET adhered cells (mean+/−SD, N=3). A two-way ANOVA with multiple comparisons was performed to determine statistical difference between conditions (*p<0.05).

### DETAILED DESCRIPTION OF THE INVENTION

The present invention relates to methods and compositions comprising an anti-CAECAM1 antibody for the treatment of NET-associated pathologies, including non-malignant thrombotic diseases and disorders, and for preventing, delaying or inhibiting metastatic formation, migration, spread and progression.

Without wishing to be bound to any theory or mechanism or action, it is proposed that inhibiting the trapping of cells by NETotic matrices, prevents pathological aggregation of cells which may lead, if not inhibited, to formation of thrombi or metastases. The present invention exemplifies the success of utilizing CM24, a humanized anti-CEACAM1 antibody, in inhibiting NET-mediated cancer cell migration in-vitro and suppressing metastasis in-vivo.

The findings disclosed in the present invention also provides a better understanding of the crosstalk between cancer and NETs, that is crucial for the development of novel therapeutic interventions blocking cancer evasion mechanisms and preventing metastatic spread.

Furthermore, the present invention describes the utility of NET markers, as effective biomarkers for selecting patients eligible for treatment with anti-CEACAM1 antibodies, and for monitoring the effectiveness of treatment with such antibodies. In a non-limiting example, the NET marker is myeloperoxidase (MPO).

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

The term “CEACAM1” is used to refer to the protein product of the human CEACAM1 gene e.g., NP_001020083.1, NP_001703.2. In humans, 11 different CEACAM1 splice variants have been detected so far. Individual CEACAM1 isoforms differ with respect to the number of extracellular immunoglobulin-like domains (for example, CEACAM1 with four extracellular immunoglobulin-like domains is known as CEACAM1-4), membrane anchorage and/or the length of their cytoplasmic tail (for example, CEACAM1-4 with a long cytoplasmic tail is known as CEACAM1-4L and CEACAM1-4 with a short cytoplasmic tail is known as CEACAM1-4S). The N-terminal domain of CEACAM1 starts immediately after the signal peptide and its structure is regarded as IgV-type. For example, in CEACAM1 annotation P13688, the N-terminal IgV-type domain is comprised of 108 amino acids, from amino acid 35 to 142. This domain was identified as responsible for the homophilic binding activity (Watt et al., 2001, Blood. 98, 1469-79). All variants, including these splice variants are included within the term “CEACAM1”.

The terms “anti-CEACAM1 antibody”, “an antibody which recognizes CEACAM1”, “an antibody against CEACAM1” and “an antibody to CEACAM1” are interchangeable, and used herein to refer to an antibody that binds to the human CEACAM1 protein with sufficient affinity and specificity.

Specific anti CEACAM1 antibodies are described in WO2010125571, that disclose, for example, a murine anti human CEACAM1 antibody denoted MRG1.

CM24 is a non-fully humanized mAb disclosed in detail in WO2015166484.

The unique properties of CM24 and similar antibodies included in the compositions and methods of the present invention, confer several advantages to their use in human, specifically in applications requiring long-term or repeated administration, when other, non-human antibodies cannot be administered in the fear of eliciting an immunogenic response towards the non-human antibodies themselves. Avoiding such an immune response becomes more crucial when the treated person is a patient inflicted with a disease, where further aggravating the patient's health should be avoided.

As used herein, the term “NET” or “NETs” refers to extracellular complexes of nucleosomes and proteins, e.g., proteins having anti-microbial activity. The extracellular complexes may be derived from any myeloid or lymphoid cell including neutrophils, macrophages, myeloid-derived suppressor cells, mast cells, eosinophils, basophils, dendritic cells, neutral killer cells, monocytes, or B- and T-cells.

Neutrophils and macrophages are the main cell types known to cast extracellular traps, composed of DNA and histones (mostly in their citrullinated form), and further decorated by different proteins. Like neutrophils, macrophages undergo cell death known as METosis, in which they cast the extracellular traps that include proteins such as MPO. (Rahat et al. Front Immunol. 2023 Sep. 26:14:1292819). Any aspect, embodiment and claim of the present invention that relate to NET, NETs, NET marker, NET-related condition, and NETosis, also encompass macrophage-derived extracellular traps, namely, MET, METs, METosis, MET marker, MET-related condition, and METosis.

The terms “NET-associated condition”, “NET-associated disease”, “NET-associated disorder”, “NET-driven disorders” and “NETopathies” are interchangeable, and used herein to refer to a pathological or condition that involves an undesired or uncontrolled NETosis process.

The terms “NET marker” is used to indicate a biological substance obtained from a subject, that is measured quantitatively or qualitatively to detect NETosis. Detecting NETosis includes but is not limited to detection of NET formation, detection comprises detection of co-localized neutrophil-derived proteins and extracellular DNA and citrullinated histones, detection of NET remnants in fluid samples, and flow cytometric detection of cell-appendant NET components. Any marker known in the art to detect NETosis may be used with the methods of the present invention. According to some embodiments, NETs markers include but are not limited to: myeloperoxidase (MPO), Neutrophil Elastase (NE), Peptidyl Arginine Deiminase 4 (PAD4), Citrullinated Histone H3 (Cit-H3) and cell free DNA. According to some embodiments, the at least one marker is selected from MPO, NE and DNA complex. According to specific embodiments, the NET marker is MPO.

The term “biomarker” in connection with NETs or NETosis is used, according to some embodiments of the present invention, to describe a diagnostic tool or prognostic tool, e.g., in patient selection.

The terms “NET marker” and NET-biomarker” are used interchangeably, according to some embodiments of the present invention.

The term control sample denotes a sample taken from a healthy subject or subjects or a sample taken from the evaluated subject being in a different (e.g., earlier, pre-treatment) stage of the disease.

The terms significantly or significant refer to differences, calculated by statistical methods known in the art, to determine that a result or an observation from a set of data is due to intrinsic qualities and not random variance of a sample.

The term “antigen” as used herein refers to a molecule or a portion of a molecule capable of eliciting antibody formation and being bound by an antibody. An antigen may have one or more than one epitope. The specific reaction referred to above is meant to indicate that the antigen will react, in a highly selective manner, with its corresponding antibody and not with the multitude of other antibodies which may be evoked by other antigens. An antigen according to the present invention is a human CEACAM1 protein or a fragment thereof. According to some embodiments, human programmed cell death 1 (PD-1) protein also serves an antigen for combined therapy according to the present invention

The term “antigenic determinant” or “epitope” as used herein refers to the region of an antigen molecule that specifically reacts with a particular antibody. Peptide sequences derived from an epitope can be used, alone or in conjunction with a carrier moiety, applying methods known in the art, to immunize animals and to produce additional polyclonal or monoclonal antibodies.

The term “antibody” is used in the broadest sense and includes monoclonal antibodies (including full length or intact monoclonal antibodies), polyclonal antibodies, multivalent antibodies, multi-specific antibodies (e.g., bi-specific antibodies), and antibody fragments that include at least the antibody binding portion and exhibit the desired biological activity, namely to CEACAM1.

The antibody according to the present invention is a molecule comprising at least the antigen-binding portion of an antibody. Antibody or antibodies according to the invention include intact antibodies, such as polyclonal antibodies or monoclonal antibodies, as well as proteolytic fragments thereof, such as the Fab or F(ab′)2 fragments. Other types of antibody fragments and constructs, and single chain antibodies also fall within the scope of the present invention.

The terms “Molecule having the antigen-binding portion of an antibody” and “antigen-binding-fragments” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)2 fragment, the variable portion of the heavy and/or light chains thereof, Fab mini-antibodies (see WO 93/15210, U.S. patent application Ser. No. 08/256,790, WO 96/13583, U.S. patent application Ser. No. 08/817,788, WO 96/37621, U.S. patent application Ser. No. 08/999,554, the entire contents of which are incorporated herein by reference), dimeric bispecific mini-antibodies (see Muller et al., 1998) and single-chain antibodies incorporating such reactive fraction, as well as any other type of molecule in which such antibody reactive fraction has been physically inserted. Such molecules may be provided by any known technique, including, but not limited to, enzymatic cleavage, peptide synthesis or recombinant techniques.

“Antibody fragments” comprise only a portion of an intact antibody, generally including an antigen binding site or portion of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 1989, 341, 544-546) which consists of a VH domain; (vii) isolated CDR regions; (viii) F(ab′)2 fragments, a bivalent fragment including two Fab′ fragments linked by a disulphide bri'ge at the hinge region; (ix) single chain antibody molecules (e.g. single chain Fv; scFv) (Bird et al., Science 1988, 242, 423-426; and Huston et al., PNAS (USA) 1988, 85,5879-5883); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 1993, 90, 6444-6448); (xi) “linear antibodies” comprising a pair of tandem Fd segments (VH-CH1-VH-CH1) which, together with complementary light chain polypeptides, form a pair of antigen binding regions (Zapata et al. Protein Eng., 1995, 8, 1057-1062; and U.S. Pat. No. 5,641,870).

Single chain antibodies can be single chain composite polypeptides having antigen binding capabilities and comprising amino acid sequences homologous or analogous to the variable regions of an immunoglobulin light and heavy chain i.e. linked VH-VL or single chain Fv (scFv).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. Monoclonal Abs may be obtained by methods known to those skilled in the art. For example, the monoclonal antibodies to be used in accordance with the present invention may be made by the hybridoma method first described by Kohler et al., Nature 1975, 256, 495, or may be made by recombinant DNA methods (see, e.g., U.S. Pat. No. 4,816,567). The “monoclonal antibodies” may also be isolated from phage antibody libraries using the techniques described in Clackson et al., Nature 1991, 352, 624-628 or Marks et al., J. Mol. Biol., 1991, 222:581-597, for example.

The mAbs of the present invention may be of any immunoglobulin class including IgG, IgM, IgE, or IgA. in particular embodiments, the mAbs of the invention are IgG.

Humanized antibodies are antibodies from non-human species (e.g. murine antibodies) whose protein sequences have been modified to increase their similarity to antibody variants produced naturally in humans. The process of “humanization” is usually applied to monoclonal antibodies developed for administration to humans, and performed when the process of developing a specific antibody involves generation in a non-human immune system (such as in mice). The protein sequences of antibodies produced in this way are distinct from antibodies occurring naturally in humans, and are therefore immunogenic when administered to human patients. Humanized antibodies are considered distinct from chimeric antibodies, which have protein sequences similar to human antibodies, but carry large stretches of non-human protein.

It is possible to produce a humanized antibody without creating a chimeric intermediate. Direct creation of a humanized antibody can be accomplished by inserting the appropriate CDR coding segments (responsible for the desired binding properties) into a human antibody scaffold, a process known as “CDR grafting”. In general, after an antibody is developed to have the desired properties in a mouse (or another non-human animal), the DNA coding for that antibody's CDRs can be sequenced. Once the precise sequences of the desired CDRs are known, these sequences are inserted into a construct containing the DNA for a human antibody framework.

CDR identification or determination from a given heavy or light chain variable sequence, is typically made using one of few methods known in the art. For example, such determination is made according to the Kabat (Wu T. T and Kabat E. A., J Exp Med, 1970; 132:211-50) and IMGT (Lefranc M-P, et al., Dev Comp Immunol, 2003, 27:55-77).

There are several methods known in the art for determining the CDR sequences of a given antibody molecule, but there is no standard unequivocal method. Determination of CDR sequences from antibody heavy and light chain variable regions can be made according to any method known in the art, including but not limited to the methods known as KABAT, Chothia and IMGT. A selected set of CDRs may include sequences identified by more than one method, namely, some CDR sequences may be determined using KABAT and some using IMGT, for example. According to some embodiments, the CDR sequences of the mAb variable regions are determined using the IMGT method.

When the term “CDR having a sequence”, or a similar term is used, it includes options wherein the CDR comprises the specified sequences and also options wherein the CDR consists of the specified sequence.

The antigen specificity of an antibody is based on the hyper variable region (HVR), namely the unique CDR sequences of both light and heavy chains that together form the antigen-binding site.

Since the variable regions of some of the antibodies included in the compositions and method of the present invention, e.g., CM24, differ in at least one amino-acid from the variable regions of fully human antibodies, they are also labeled “non-fully-humanized” antibodies. The term “non-fully-humanized monoclonal antibody” as used herein thus refers to a monoclonal antibody, having a heavy chain and/or a light chain variable domains in which the amino-acid sequences flanking and/or immediately adjacent to the CDRs are not fully human, i.e. are not identical to any known homologous or corresponding sequences taken from natural human antibodies.

An antibody sequence which contains a substitution, deletion and/or insertion of at least one amino-acid, and up to about 10% of a chain sequence, in comparison to a respective sequence is also included within the scope of the present invention. These substitutions are typically made within a “non-CDR sequence”, namely, a sequence of the constant domains or a sequence comprised in a variable region of an antibody, which is not a CDR sequence as disclosed above. Alterations in the CDR sequences are less common but are also permitted as long as the antibody binding is not affected.

The terms “homologous”, “homology”, “precent identity”, or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990).

The invention also provides conservative amino acid variants of the antibody and antibody fragments specifically disclosed. Variants according to the invention also may be made that conserve the overall molecular structure of the encoded proteins. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved. The term “antibody analog” as used herein refers to an antibody derived from another antibody by one or more conservative amino acid substitutions.

Antibody variants and conjugates, referring to any molecule comprising the antibody of the present invention. For example, fusion proteins in which the antibody or an antigen-binding-fragment thereof is linked to another chemical entity is also within the scope of the present invention.

In some embodiments, an antibody provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or less (e.g., 10⁻⁸ M or less, e.g., from 10⁻⁸ M to 10⁻¹³ M, e.g., from 10⁻⁹ M to 10⁻¹³ M) for human CEACAM1. K_{D} can be measured by any suitable assay. In certain embodiments, KD can be measured using surface plasmon resonance (SPR) assays (e.g., using a BIACORE® instrument).

The present invention is based in part on the results of a clinical trial conducted to evaluate the safety and efficacy of the anti CEACAM1 mAb CM24 (NCT04731467). Part A of the trial assessed the safety of CM24 administration and involved administration of CM24 in combination with the anti-PD1 mAb Nivolumab, in dose escalation, to patients with solid tumors. Part C also assessed safety, and consisted of two subparts-part C1, during which 8 pancreatic cancer patients were administered CM24 in combination with Nivolumab, and an additional therapeutic agent selected from gemcitabine and nab-paclitaxel; and part C2, in which 8 additional pancreatic cancer patients were administered CM24 in combination with Nivolumab, and the chemotherapeutic cocktail Nal-IRI/5FU/LV. Part D, the final stage of the clinical trial, assesses the efficacy of the treatment, by monitoring the progression of the patients following treatments described above, compared to control patients who were administered standard therapy (gemcitabine/nab-paclitaxel or Nal-IRI/5FU/LV) and did not receive antibody therapy. As used herein, “second-line therapy”, refers to a subsequent therapeutic regimen following an unsuccessful or insufficient initial therapeutic regimen. Second-line therapy can comprise the same therapeutic agents administered to the patient the first time, or entirely different ones. In pharmaceutical and medicament formulations, the active agent is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired daily dose.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

The pharmaceutical compositions may alternatively be formulated to control release of active ingredient (molecule comprising the antigen binding portion of an antibody) or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention, i.e., of an antibody or antibody fragment, from such a matrix depends upon the molecular weight of the molecule, the amount of the molecule within the matrix, and the size of dispersed particles.

According to some embodiments, the pharmaceutical composition comprises 1-50 mg/ml of humanized mAb to CEACAM1, e.g., CM24. According to some embodiments, the pharmaceutical composition comprises a basic amino acid. According to some embodiments, the pharmaceutical composition comprises a sugar. According to some embodiments, the pharmaceutical composition comprises a surfactant. According to some embodiments, the pharmaceutical composition comprises a basic amino acid, a sugar and a surfactant. According to some embodiments, the pharmaceutical composition comprises (i) 1-10 mg/ml of basic amino acid; (ii) 10/100 mg/ml of a sugar; (iii) 0.01-1 mg/ml of a surfactant; (iv) 1-50 mg/ml of humanized mAb to CEACAM1 (e.g., CM24), 4-6 mg/ml of basic amino acid, 70-100 mg/ml of a sugar and a 0.1-1 mg/ml of non-anionic surfactant; or (v) 10 mg/ml of CM24, 4.65 mg/ml of L-Histidine, 82 mg/ml of sucrose and 0.20 mg/ml of polysorbate 20.

The term “sugar” refers to monosaccharides, disaccharides, and polysaccharides, Examples of sugars include, but are not limited to, sucrose, trehalose, dextrose, and others.

According to some embodiments, the basic amino acid is selected from the group consisting of: Histidine, Arginine, Lysine and Ornitine. Each possibility represents a separate embodiment of the present invention. According to some embodiments, the composition comprises 1-10, 2-9, 3-7 or 4-6 mg/ml of basic amino acid. Each possibility represents a separate embodiment of the present invention.

According to some embodiments, the sugar is selected from the group consisting of: sucrose, trehalose, glucose, dextrose and maltose. Each possibility represents a separate embodiment of the present invention. According to some embodiments, the composition comprises 10-200, 10-100, 50-150 or 70-100 mg/ml of sugar. Each possibility represents a separate embodiment of the present invention.

According to yet other embodiments, the composition comprises polyol, including but not limited to mannitol and sorbitol. Each possibility represents a separate embodiment of the present invention.

According to some embodiments, the surfactant is a non-anionic. According to some embodiments, the surfactant selected from the group consisting of: polysorbates, sorbitan esters and poloxamers. Each possibility represents a separate embodiment of the present invention. According to some embodiments, the surfactant selected from the group consisting of: polysorbate 20, polysorbate 80. Each possibility represents a separate embodiment of the present invention. According to some embodiments, the composition comprises 0.01-10, 0.01-1, 0.05-5 or 0.1-1 mg/ml of surfactant. Each possibility represents a separate embodiment of the present invention. According to some embodiments, the pharmaceutical composition comprises 4-6 mg/ml of basic amino acid, 70-100 mg/ml of a sugar and a 0.1-1 mg/ml of surfactant.

According to some embodiments, the pharmaceutical composition is in a liquid form and comprises 1-50 mg/ml of CM24. According to other embodiments, the pharmaceutical composition is lyophilized. According to some embodiments, the pharmaceutical composition comprises: 10 mg/ml of CM24, 4.65 mg/ml of L-Histidine, 82 mg/ml of sucrose and 0.20 mg/ml of polysorbate 20.

According to some embodiments, the pharmaceutical composition comprises CM24 or a fragment defined above, and an additional immuno-modulator or a kinase inhibitor. According to some embodiments, a pharmaceutical composition comprising at least one humanized mAb or fragment defined above, and a pharmaceutical composition comprising an additional immuno-modulator or a kinase inhibitor, are used in treatment of cancer by separate administration.

According to some specific embodiments, the additional immuno-modulator is selected from the group consisting of: an anti-human programmed cell death protein 1 (PD-1), PD-L1 and PD-L2 antibody, an activated cytotoxic lymphocyte cell, a lymphocyte activating agent, and a RAF/MEK pathway inhibitor. Each possibility represents a separate embodiment of the present invention. According to some specific embodiments, the additional immuno-modulator is selected from the group consisting of: mAb to PD-1, mAb to PD-L1, mAb to PD-L2, Interleukin 2 (IL-2), lymphokine-activated killer (LAK) cell.

The pharmaceutical composition of this invention may be administered by any suitable means, including parenteral and enteral routs. Administration modes include but are not limited to, oral, topical, intranasal, subcutaneous, intramuscular, intravenous, intra-tumoral, intra-arterial, intraarticular, intralesional and transdermal. Ordinarily, intravenous (i.v.), administration is used.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician. As used herein, a “therapeutically effective amount” refers to the amount of a molecule required to alleviate one or more symptoms associated with a disorder being treated over a period of time.

As used herein the term “individual,” “patient,” or “subject” refers to individuals diagnosed with, suspected of being afflicted with, or at-risk of developing at least one disease for which the described compositions and method are useful for treating. According to some embodiments the individual is a mammal. According to some embodiments, the mammal is a mouse, rat, rabbit, dog, cat, horse, cow, sheep, pig, goat, llama, alpaca, or yak. According to some embodiments, the individual is a human.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic or being conjugated to a cytostatic and/or cytotoxic agent. For cancer therapy, efficacy in vivo can, for example, be measured by assessing the duration of survival, time to disease progression, the response rates, duration of response, and/or quality of life.

In some embodiments, administration of an anti CEACAM1 antibody for treatment of cancer or prevention of cancer metastases, comprises at least one additional anti-cancer therapy. According to some embodiments, the at least one additional anti-cancer therapy is selected from the group consisting of chemotherapy, radiation, surgery, and immunotherapy.

As used herein the term “combination” or “combination treatment” can refer either to concurrent administration of the articles to be combined or sequential administration of the articles to be combined. As described herein, when the combination refers to sequential administration of the articles, the articles can be administered in any temporal order.

As used herein “checkpoint inhibitor” refers a drug that inhibits a biological molecule (“checkpoint molecule”) produced by an organism that negatively regulates the anti-tumor/cancer activity of T cells in the organism. Checkpoint molecules include without limitation PD-1, PD-L-1, PD-L-2, CTLA4, TIM-3, LAG-3, VISTA, SIGLEC7, PVR, TIGIT, IDO, KIR, A2AR, B7-H3, B7H4, and CD112R.

According to some embodiments, the method of treating cancer comprises administering the pharmaceutical composition as part of a treatment regimen comprising administration of at least one additional anti-cancer agent. According to a specific embodiment the anti-cancer composition comprises at least one chemotherapeutic agent. The chemotherapy agent, which could be administered together with the antibody according to the present invention, or separately, may comprise any such agent known in the art exhibiting anticancer activity.

The terms “anti-cancer” and “anti-neoplastic composition” refers to a composition useful in treating cancer comprising at least one active therapeutic agent capable of inhibiting or preventing tumor growth or function, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells and immune checkpoint inhibitors.

According to a specific embodiment, the chemotherapeutic agent is selected from the group consisting of alkylating agents, antimetabolites, folic acid analogs, pyrimidine analogs, purine analogs and related inhibitors, vinca alkaloids, epipodophyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), Irinotecan (including Nal-Irinotecan), oxaliplatin, capecitabine, paclitaxel and doxetaxel. Two or more chemotherapeutic agents can be used in a cocktail to be administered in combination with administration of the anti-CEACAM1 antibody. According to some embodiments, chemotherapeutic cocktail comprises Nal-Irinotecan/5FU/LV.

The term “treatment” as used herein refers to both therapeutic treatment and prophylactic or preventative measures. Those in need of treatment include those already with the disorder as well as those in which the disorder is to be prevented. This term includes preventative treatment of subjects that are predisposed to a disease or disorder, e.g., subject genetically or environmentally predisposed to cancer or to another disease. In some embodiments, a course of treatment is preceded by the acquisition of a biological sample from the subject. A biological sample is any sample collected from the individual, and including but not limited to samples of a fluid (such as blood, urine, saliva or cerebrospinal fluid), and tissue, acquired by biopsy, surgical removal or resection of a part of a limb, or a benign or metastatic growth.

In certain embodiments, the method described above comprises administering to the subject at least one dose of a CM24 ranging from 0.01 mg/kg to 10 mg/kg body weight. In certain embodiments, the method described above comprises administering (i) multiple, identical, or different, doses of CM24; (ii) multiple escalating doses; or (iii) the pharmaceutical composition once every week, one every 2 weeks, once every 3 weeks, once every 4 weeks, or once every 5 weeks. In certain embodiments, the method described above comprises 1-10 administration cycles, each cycle comprising 2-5 infusions every 1-4 weeks, with CM24, followed by a 2-8 weeks between each cycle. In certain embodiments, the method described above comprises administering CM24 as neoadjuvant, adjuvant, or maintenance treatment.

The terms “cancer”, “cancerous” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

Examples of cancer include but are not limited to, carcinoma, lymphoma, blastoma, sarcoma, and leukemia. More particular examples of such cancers include melanoma, lung, thyroid, breast, colon, prostate, hepatic, bladder, renal, cervical, pancreatic, leukemia, lymphoma, myeloid, ovarian, uterus, sarcoma, biliary, or endometrial cancer. In certain embodiments, the cancer comprises a solid tumor. In certain embodiments, the cancer is selected from the group consisting of colon cancer, pancreatic cancer, breast cancer, bladder cancer, kidney cancer, head and neck cancer, ovarian cancer, glioblastoma, cervical cancer, prostate cancer, and lung cancer. In other embodiments, the cancer is selected from the group consisting of: melanoma, colorectal, bladder, lung, non-small cell lung carcinoma (NSCLC), non-small cell lung adenocarcinoma (NSCLA), gastrointestinal, pancreatic, breast, prostate, thyroid, stomach, ovarian, myeloma and uterine cancer. Each possibility represents a separate embodiment of the present invention.

According to some embodiments, the cancer comprises a solid tumor. According to some embodiments, the cancer is a metastatic cancer or tumor.

According to some embodiments, the solid cancer is a pancreatic cancer, a lung cancer, a colorectal cancer or melanoma. According to some embodiments the cancer is a metastatic cancer initiated from a primary pancreatic, lung, colorectal or melanoma tumor.

NETs provide a scaffold and stimulus for thrombus formation. Release of NETs from neutrophils has been associated with inflammation during sepsis and noninfectious diseases. Accordingly, as described herein, thrombotic conditions can be treated or prevented by the disruption of NETs.

As used herein, the phrase “cardiovascular condition” or “cardiovascular disease or disorder” is intended to include all disorders characterized by insufficient, undesired or abnormal cardiac function, e.g., ischemic heart disease, hypertensive heart disease and pulmonary hypertensive heart disease, valvular disease, congenital heart disease and any condition which leads to congestive heart failure in a subject, particularly a human subject. Also included are any diseases and conditions of the blood vessels which result in insufficient, undesired, or abnormal cardiac function e.g. stroke, thrombosis, ischemia, ischemic reperfusion, vessel occlusion, inflammation etc. As used herein, cardiovascular condition is not limited to those conditions resulting from artherosclerosis. Insufficient or abnormal cardiac function can be the result of disease, injury and/or aging. In certain embodiments, the methods and compositions provided herein are directed to the treatment or prevention of cardiovascular conditions caused or contributed to NETs activity by administering to a patient an effective dose of a composition comprising anti-CEACAM1 antibody. In a further embodiment the cardiovascular condition is stroke, ischemic reperfusion, myocardial infarction, inflammation, or thrombosis. In certain embodiments, the cardiovascular condition to be treated is thrombosis. Clinically, inflammation and infection are linked to thrombosis. Accordingly, some embodiments provide methods and compositions for treating or preventing thrombosis in a patient, e.g., methods for treating or preventing cardiovascular conditions complicated by thrombosis. Thrombosis is the occurrence of a clot in a blood vessel at a site of injury to the vessel or an inappropriate blood clot in a blood vessel and depends on the adhesion, activation, and aggregation of platelets. Deep vein thrombosis (DVT) is often linked to inflammation and infections. A complication of thrombosis is that the clot will detach from the blood vessel wall where it formed and lodge somewhere else in the circulatory system, blocking blood flow and causing an embolism. In certain embodiments, the cardiovascular condition to be treated is ischemia. In another embodiment, the cardiovascular condition to be treated is ischemic reperfusion. As used herein, the term “ischemia” refers to any localized tissue ischemia due to reduction of the inflow of blood. The flow of blood to a tissue can be reduced due to an abnormality in the blood vessels such as thrombosis, embolism, or vasoconstriction. The reduced flow of blood results in local anemia, reduced oxygen levels and eventually damage to the tissue. Ischemia can also be caused by myocardial infarction, acute coronary syndrome, coronary artery bypass surgery, stroke, gastrointestinal ischemia, peripheral vascular disease, and surgical procedures. Further, the recruitment of leukocytes and/or platelets triggered by the original tissue damage can restrict blood flow in smaller capillaries, resulting in a second wave of ischemia. The term “myocardial ischemia” refers to a subset of ischemia that encompasses circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue. In certain embodiments, the cardiovascular condition to be treated is myocardial infarction. A myocardial infarction (i.e., a heart attack) is the death of heart muscle from the sudden blockage of a coronary artery by a blood clot. Coronary arteries are blood vessels that supply the heart muscle with blood and oxygen. Blockage of a coronary artery deprives the heart muscle of blood and oxygen, causing injury to the heart muscle. Injury to the heart muscle causes chest pain and pressure. Inflammation is known to contribute to development of a myocardial infarction, particularly via formation of atherosclerotic plaques. Disruption of a plaque can cause thrombosis and lead to myocardial infarction. In certain embodiments, the cardiovascular condition to be treated is stroke. Thromboembolic occlusion of intracerebral arteries restricts downstream blood flow, promoting second arythrombi formation within the cerebral microvasculature. In certain embodiments, the cardiovascular condition to be treated is thrombosis and the patient has systemic lupus erythematosus (SLE). SLE patients are also prone to develop venous thrombosis and have a reduced ability to degrade NETs. In certain embodiments, the condition to be treated is sickle cell disease (SCD) is a condition in which RBCs are deformed and rigid. The altered RBCs are more likely to restrict blood flow at certain points in the circulatory system, leading to a crisis. In SCD patients, a lethal crisis is often precipitated by an infection.

In some embodiments, the formation of a deep vein thrombosis (DVT) is prevented or inhibited. In some embodiments, the progression of one or more signs or symptoms of DVT is prevented or inhibited, e.g., a thrombus does not increase in size. In some embodiments, the severity of one or more signs or symptoms of DVT is decreased, e.g. a thrombus decreases in size.

In another aspect, the methods described herein relate to inhibition of the formation of NETs in a subject, comprising administering to a patient an effective dose of anti-CEACAM1 antibody. In some embodiments, inhibiting the formation of NETs can comprise preventing the formation of a NET and/or reducing the likelihood that a NET will form in a subject. In some embodiments, inhibiting the formation of NETs can comprise inhibiting the growth or progression of pre-existing NETs and/or reducing the likelihood that a pre-existing NET will grow or progress in a subject. In some embodiments, the method of inhibiting the formation of NETs can reduce the severity of symptoms associated with the development of NETs, e.g. thrombosis. In some embodiments, a subject receiving treatment to inhibit the formation of NETs can be a subject having or diagnosed as having a cardiovascular condition as described above. In some embodiments, a subject receiving treatment to inhibit NET-mediated activity can be a subject having or diagnosed as having a condition which makes the subject predisposed to thrombosis (i.e., prothrombotic). A condition which makes the subject prothrombotic can be any condition in which the subject is more likely to have or to form NET-mediated thrombosis as compared to a healthy subject.

The widely accepted cross talk between inflammation and thrombosis has led to the introduction of the term thromboinflammation. Cells of the hematopoietic system, including neutrophils, platelets, and monocytes, have a major role in this process. There is increasing evidence implicating NET release with the development of both vein and arterial thrombosis. The methods and compositions of the present invention may be used for these conditions.

The role of NETs in the pathogenesis of autoimmune diseases was previously suggested. Prolonged exposure to NETs-related cascades is associated with autoimmunity and increases the chance of systemic organ damage. Several autoimmune and immune-mediated diseases are NET-associated and therefore eligible for treatment using the compositions and methods of the present invention. This includes, for example, at least one of the following disease characteristics: increased NET formation, correlation between the number of NETotic disease severity or progression, induction of NET by disease auto-antibodies, enhancement of procoagulant activity, stimulation of neutrophils to produce NETs.

The NET-associate autoimmune and immune-mediated diseases include but are not limited to: psoriasis, systemic lupus erythematosus (SLE), antiphospholipid antibody syndrome, rheumatoid arthritis (RA), ulcerative colitis (UC), gout, ANCA-associated vasculitis, dermatomyositis, and polymyositis.

Autoinflammatory disorders, such as hereditary Familiar Mediterranean fever (FMF) are also included within the legible diseases and conditions of the present invention. FMF characterized by inflammatory attacks and neutrophil infiltration into the affected sites. During FMF attacks, neutrophils undergo excessive NET formation, which decreases after the inflammation dissolution. These, and other autoimmune and autoinflammatory disorders are eligible for treatment with the compositions and methods of the present invention.

Several systemic inflammatory responses and diseases are also eligible for treatment with the compositions of the present invention. These conditions include, but are not limited to, sepsis (septic shock) in which functionally active tissue factor (TF) is found on peripheral NETs; and disseminated intravascular coagulation (DIC) in which NETs are associated with venous thromboembolism (VTE) and impaired fibrinolysis.

Several inflammatory lung diseases are characterized by the migration and detection of neutrophils and monocytes in the airway lumen and the bronchoalveolar lavage fluid. NETs have been associated with inflammatory diseases, such as chronic obstructive pulmonary disease (COPD), cystic fibrosis (CF), acute lung injury, acute respiratory distress syndrome, and asthma. These and other inflammatory diseases are also included within the scope of conditions eligible for treatment according to the present invention.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Examples

### Example 1: Effect of NETs and CM24 on Migration of Cancer Cells

TABLE 1
Materials

| Material | Vendor | Item No. | Lot No. |
|---|---|---|---|
| SKMEL-28 | ATCC | HTB-72 | N/A |
| A549 | ATCC | CRM-CCL-185 | N/A |
| AsPC-1 | ATCC | CRL-1682 | N/A |
| PBS | Corning | 21-040-CM | 29320010 |
| PMA (1 mM stock) | Cayman | 10008014 | 0584091-17 |
| NETs | Generated in-house | | |
| | with PMA | | |
| hIgG4 | BioXCell | BE0349 | 82132101 |
| Trypsin | Corning | 25-053-Cl | 17621003 |
| Accutase | Innovative Cell | AT-104 | 1A2005A |
| | Tech | | |
| CFDA-SE | Cayman | 14456 | 0587471-11 |
| CytoTrace Red | Cayman | 20698 | 0636212-1 |
| CMPTX | | | |
| Fluoroblok | BD Falcon | 351164 | 084502B |
| migration plates | | | |

### Cell Culture

Human melanoma cell line SKMEL-28, human lung adenocarcinoma cell line A549, and human pancreatic adenocarcinoma cell line AsPC-1 were cultured according to ATCC recommendations and lifted for passaging and assays with trypsin.

### Migration Assay

Cells were expanded for up to 80% confluency for two passages. The day before treatment, medium containing serum was removed and serum-free medium was added. The next day, cells were lifted and stained with 5 μM carboxyfluorescein succinimidyl ester (CFDA-SE) dye for 10 minutes at 37° C. Stained cells were washed twice with serum-containing medium to ensure excess stain was inactivated, and cells were resuspended in serum-free medium and added to the top chambers of Fluoroblok 96 well plates. Melanoma SKMEL-28 cells were added at 12,500 cells/well, NSCLC A549 cells were added at 25,000 cells/well and pancreatic cancer AsPC1 cells. NETs were prepared according to Rayes et al. ibid and added to top chambers at a final concentration of 20 ng/μl. hIgG4 (as isotype control) and CM24 were added to appropriate wells of plate at a final concentration of 500 μg/ml in serum free media. Serum-free medium or 10% serum containing medium was added to the feeder wells of the plate through the sample ports. The plate was transferred to cell imaging multimode reader (Cytation 5) with CO₂ and a 37° C. setpoint and imaged using brightfield and GFP cubes at 10× every 2 hours for a total of 24 hours.

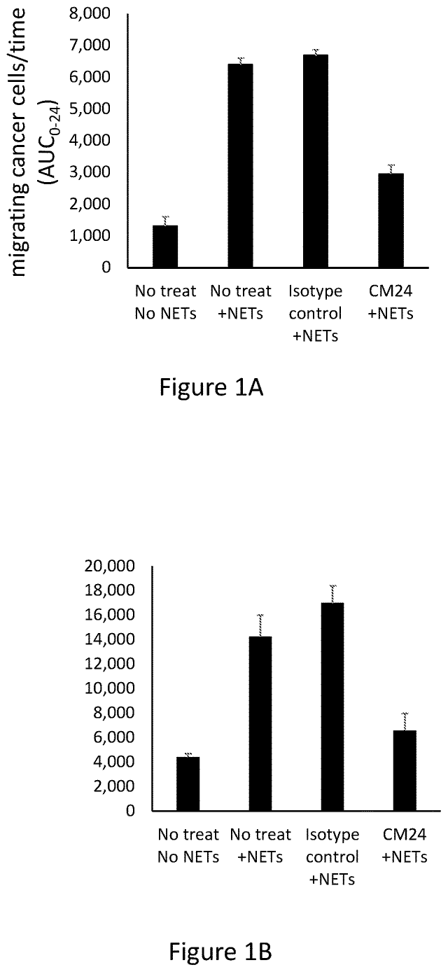

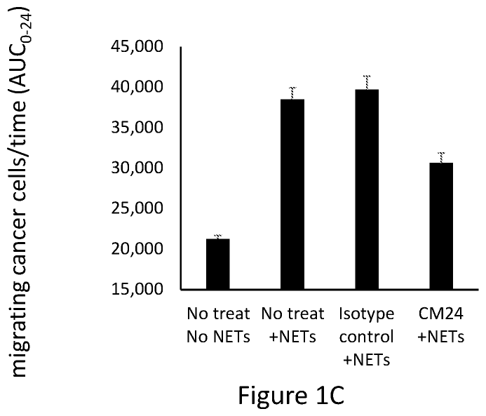

NET-induced migration was tested in an assay for cellular chemotaxis. In this assay, serum-starved, CFSE-labeled cancer cells were plated in the top chamber of a Fluoroblok migration plate, and full serum medium were placed in the bottom chamber. Treatments (NETs at 20 ng/μl and antibodies at 500 μg/ml) were included in the top chamber with the cells. Images of the bottom of the membrane were captured every 2 hours for 24 hours (SKMEL-28, A549) or 48 hours (AsPC-1), and total cell count of the migrating cells was calculated over time (FIGS. 1A-1C). The NET-induced migration was calculated by reducing the basal migration in the absence of NET from the total migrating cancer cells in the presence of NET, toward the chemotactic stimulus. The effect of CM24 on NET-induced migration was measured in the presence of NETs, with or without isotype control human anti-IgG4 or CM24, as described above, and the area under curve (AUC) was calculated for each well (N=6 wells for SKMEL-28 and A549; N=3 wells for AsPC-1 studies). Statistical analysis using two-way ANOVA with multiple comparisons served to test the significance of the difference between the treatment groups. Table 2 shows the percent inhibition of NET-induced migration induced by CM24 vs the isotype control.

TABLE 2
Percent inhibition of cancer cell migration (AUC of cell count) with CM24

| | human melanoma | | | | human | | | | human | | | |
|---|---|---|---|---|---|---|---|---|---|---|---|---|
| | SKMEL-28 | | | | NSCLC A549 | | | | PDAC AsPC-1 | | | |
| | Mean | Norm. | Stdev | | Mean | Norm. | Stdev | | | Norm. | Stdev | |
| | AUC | AUC^{#} | (%) | N | AUC | AUC^{#} | (%) | N | AUC | AUC^{#} | (%) | N |
| Basal | 1,313 | 0 | — | 6 | 4,415 | 0 | — | 6 | 21,216 | 0 | — | 3 |
| Migration | | | | | | | | | | | | |
| (no NETs) | | | | | | | | | | | | |
| Non-treated + | 6,406 | 5,093 | 3.0 | 6 | 14,249 | 9,834 | 12.3 | 6 | 38,496 | 17,280 | 3.8 | 3 |
| NETs | | | | | | | | | | | | |
| Isotype | 6,688 | 5,375 | 2.6 | 6 | 16,982 | 12,567 | 8.2 | 6 | 39,705 | 18,489 | 4.2 | 3 |
| hIgG1 + NETs | | | | | | | | | | | | |
| CM24 + NETs | 2,954 | 1,641 | 9.5 | 6 | 6,584 | 2,169 | 21.0 | 6 | 30,622 | 9,406 | 4.1 | 3 |
| % Inhibition of | 69.5% | | | | 82.7% | | | | 49.1% | | | |
| NET-induced | | | | | | | | | | | | |
| migration (vs. | | | | | | | | | | | | |
| isotype control) | | | | | | | | | | | | |

As shown in FIGS. 1A-1C and in Table 2, a significant increase in the cancer cell migration was observed when NETs were added to the upper wells. Addition of the isotype control had no significant effect compared with non-treated wells. However, NET-induced migration of all three cancer cell lines was significantly inhibited by CM24 (500 μg/ml) as compared to the isotype control treatment (hIgG4 500 μg/ml) or to the non-treated cells. In melanoma SKMEL-28 cells (FIG. 1A), 70% inhibition of the NET-induced migration was observed with p-value of 0.0197. In NSCLC A549 cells, CM24 inhibited the NET-induced migration by 83% (FIG. 1B), with p value<0.0001. Lastly, in the pancreatic cancer AsPC-1 cells, a significant inhibition of the NET-induced migration by 49% (p value<0.0001) was observed following treatment with CM24 as compared to the isotype control (FIG. 1C). It is therefore concluded that CM24 significantly blunts the cancer cell migration in response to NETs.

### Example 2: The Binding of CM24 to NETs

TABLE 3
Materials

| Material | Vendor | Item No. | Lot No. |
|---|---|---|---|
| Anti-MPO | ThermoFisher | PA5-16672 | XD3550635A |
| MRG1 | Provided by client | | |
| CM-24 | Provided by client | | |
| Alpaca anti-rabbit | Jackson | 611-545-215 | 159677 |
| AF488 | Immunoresearch | | |
| Donkey | Jackson | 715-116-150 | 159003 |
| anti-mouse-PE | Immunoresearch | | |
| Donkey | Jackson | 709-116-149 | 159843 |
| anti-human-PE | Immunoresearch | | |
| NETs | Generated in-house with PMA | | |
| hIgG4 (S228P) | BioXCell | CP147 | 821321D1 |
| Rabbit IgG | R&D Systems | AB105-C | ER1721081 |
| Mouse IgG1 | R&D Systems | MAB002 | 1X2721082 |
| HT-29 | ATCC | HTB-38 | 70035209 |
| AsPC-1 | ATCC | CRL-1682 | 70038492 |

| Buffer | Composition |
|---|---|
| Wash buffer | PBS + 1% BSA + 0.1% Tween20 |
| Blocking buffer | PBS + 0.1% Tween + 1% BSA + 5% goat serum |
| HT-29 media | McCoy's 5a Medium Modified (ATCC #30-2007) + |
| | 10% FBS |
| AsPC-1 media | RPMI-1640 (ATCC #30-2001) + 10% FBS |

### Sample Preparation & Immunofluorescence

Chamber slides were coated with poly-D-lysine at room temperature, and then washed well with water. Fresh neutrophils were plated in poly-D-lysine coated chamber slides (50K per chamber) and stimulated with 100 nM Phorbol-12-myristate-13-acetate (PMA) for 4 hours to induce NETosis. Medium was removed, 4% paraformaldehyde (PFA) was added and incubated for 10 minutes at room temperature. Supernatant was removed, then washed 2 times with wash buffer. Blocking buffer was added and incubated for 1 hour at room temperature. Blocking buffer was removed, the following primary antibodies were added in wash buffer, and incubated for 1 hour at room temperature. a. rabbit polyclonal anti-myeloperoxidase (MPO) 1:200 b. hIgG4 humanized mAb CM24 20 μg/ml c. human IgG4 isotype control at 20 μg/ml. Washing was done 3 times with wash buffer. Then, Secondary antibodies (a. Anti-rabbit AF488 1:600 b-c. Anti-human PE 1:200) were added in wash buffer and incubated for 1 hour at room temperature. Washing was done 3× with wash buffer. The fluorescent stain 4′,6-diamidino-2-phenylindole (DAPI) (1×) was added in PBS and imaging was done by confocal microscopy with the following LED/filter sets: a. green fluorescent protein (GFP): rabbit polyclonal anti-myeloperxodase (MPO) b. red fluorescent protein (RFP) phycoerythrin (PE): CEACAM1 c. DAPI. With chamber slides, supernatant was aspirated, then chamber parts were removed and mounted with ProLong AntiFade mountant and glass coverslips. Slides were imaged using a Nikon A1 confocal with a Ti eclipse microscope and a Nikon 40× Plan Fluor Oil DIC H N2 objective. Images were analyzed using NIS elements version 5.12.03 and ImageJ Fiji.

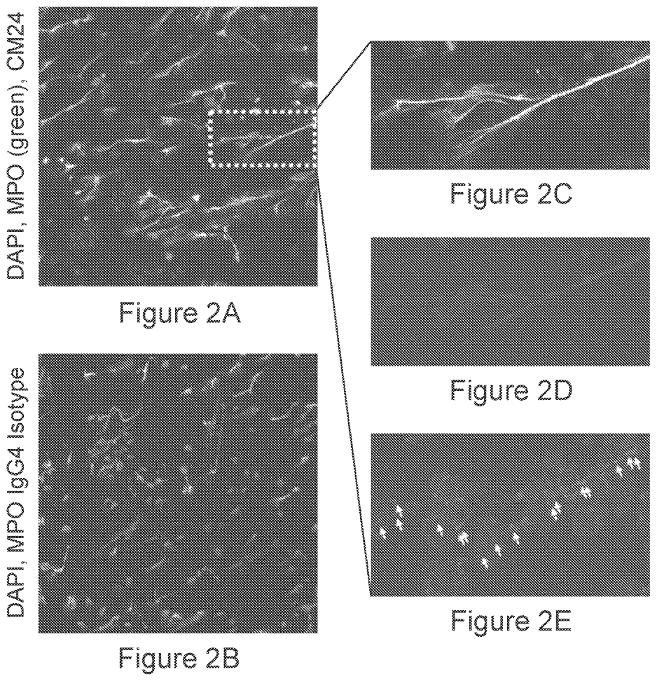

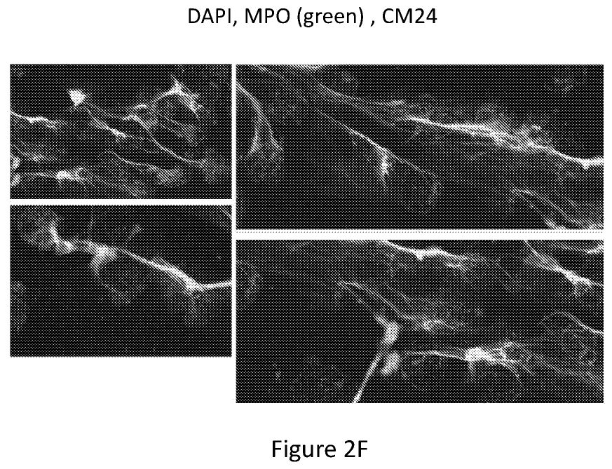

Briefly, primary human neutrophils were stimulated with PMA to undergo NETosis in poly-D lysine coated chamber slides for 4 hours, and then fixed with paraformaldehyde. These slides were stained with anti-MPO (myeloid peroxidase, a NET marker) and DAPI for extracellular DNA in the NET structure as well as nuclear DNA (in intact neutrophil), with either CM24 or isotype control, and imaged using confocal microscopy. MPO and DAPI demonstrated the typical NET morphology (FIG. 2A). Specific binding of CM24 to the NET structure was observed, as compared to the isotype control, which showed no detectable staining (FIG. 2B).

High quality images of NETs were generated when NETs were induced in chamber slides and stained directly. MPO was used as a biomarker of NETs, as it decorates the DNA structure of the NETs. CM24 was tested for binding to NETs, and specificity was assured by the parallel staining with an isotype control antibody hIgG4. Using confocal microscopy, a clear and specific binding of CM24 to CEACAM1 on NET was observed, suggesting involvement in NET-related diseases. The pattern of binding was suggestive of punctate surface staining of neutrophils undergoing NETosis with significant distribution to NETs themselves.

### Example 3: Expression of CEACAM1 on Cancer Cell Lines

In order to assess the binding potential of cancer cells to NETs through CEACAM1 interactions, the expression of CEACAM1 on different cancer cell lines, used for the migration assays (Example 1) and for the adhesion assay (Example 7), was measured. The binding of the mouse anti human CEACAM1 mAb MRG1 (described in WO2010125571) to the surface of several cell lines was tested by flow cytometry. Live cancer cells grown under normal culture conditions (e.g., maintaining less than 80% confluency) were stained with either mouse anti human CEACAM1 antibody MRG1 or isotype control followed by anti-mouse-PE. Samples were then stained for viability and run through a MACSQuant flow cytometer. Data were analyzed using FlowJo, and percentage of CEACAM1-positive cells and the intensity of CEACAM1 expression values for each cell line are shown in Table 4.

TABLE 4
Frequency (%) and mean fluorescence intensity
(MFI) of cancer cell lines stained with MRG1.

| | % CEACAM1⁺ | Anti CEACAM1 |
|---|---|---|
| Cell line | cells of live cells | binding (MFI) |
| Melanoma SKMEL-28 | 84.8 | 48,115 |
| NSCLC A549 | 73.1 | 566 |
| Pancreatic cancer AsPC1 | 99.9 | 11,688 |
| Leukemic T-cell Jurkat | 2.8 | 36 |
| (control) | | |

Jurkat cells (leukemic T-cell line), which do not express CEACAM1, were used as a negative control. Human melanoma SK-MEL-28 cell line, human pancreatic cancer AsPC-1 cell line and non-small cell lung carcinoma cells A549 cells tested in the migration assay, showed to expressed CEACAM1, detected by MRG1 while Jurkats were negative. Both SK-MEL-28 and AsPC-1 cells demonstrated high extent and expression levels of CEACAM1, where 85% and nearly 100% of the cells were positive for CEACAM1, with mean MFI (a measure of the amount of antibody bound) of 48K and nearly 12K, respectively. The majority (73.1%) of A549 cells stained positively with MRG1, with a substantially lower intensity (MFI of 566). The expression of CEACAM1 in A549 depends on cell confluency, which was similar in all studies. Without being bound to any theories, anti CEACAM1 antibody may interfere with the interaction between NET-bound CEACAM1 and CEACAM1 on cancer cells or other cells, and therefore affect NET-related diseases.

### Example 4: Clinical Trial Results Showing Decrease in MPO Levels in CM24 and Nivolumab-Treated Patients

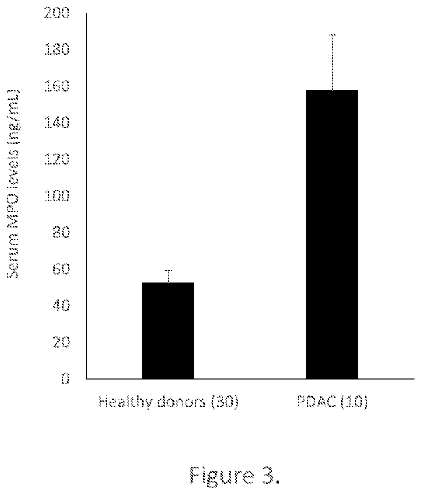

Serum samples of patients treated with CM24 and Nivolumab (clinical trial NCT04731467), were collected before and after treatment and tested for levels of the NET marker MPO. Pre-dose serum levels of the NET marker MPO in PDAC and CRC patients (n=12) were >3.5-fold higher compared to healthy subjects (n=30) (p<0.001). PDAC patients (n=10) showed 3-fold higher serum MPO compared to healthy subjects (FIG. 3).

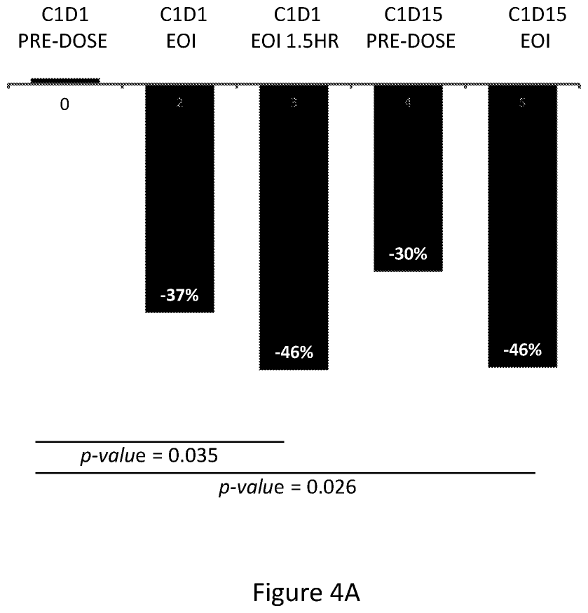

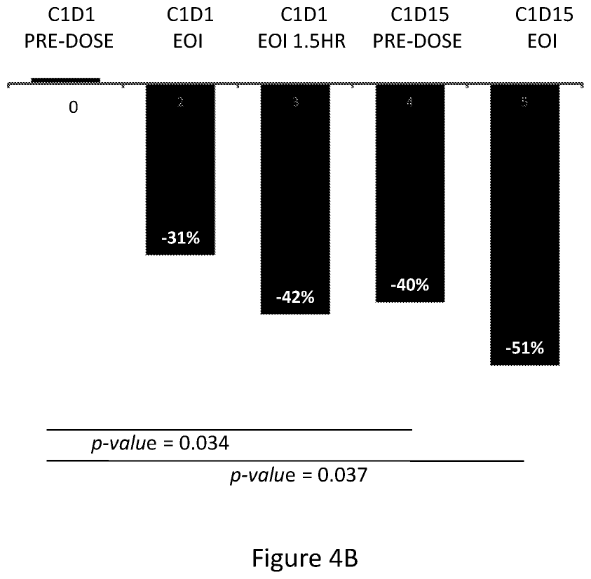

The mean results of 12 patients, depicted in FIG. 4A, show a significant decrease in serum MPO levels following treatment with CM24 both on Day 1 and Day 15 of the trial (p<0.05). FIG. 4B illustrates the same measurement of serum NET levels following CM24 treatment in PDAC patients only (n=10), demonstrating a significant decrease in serum MPO two weeks following CM24 (p<0.05).

In a dose escalation arm of the trial, adult subjects with selected recurrent or metastatic solid tumors, were treated with CM24 at doses of 10 mg/kg (3 patients), 15 mg/kg (3 patients), and 20 mg/kg (5 patients) by 1-hour infusion, followed by 30-minutes infusion of Nivolumab 480 mg/patient, every two weeks. Blood samples were collected on Day1 before treatment (C1D1 PRE-DOSE), at the end of CM24 administration (C1D1 EOI) and 1.5 hour later at the end of Nivolumab administration (C1D1 EOI 1.5 HR). Two weeks later (Day 15), blood samples were collected before treatment (C1D15 PRE-DOSE) and at the end of CM24 administration (C1D15 EOI). Serum samples were prepared immediately following each blood collection and stored in aliquots at −70° C.

NET serum levels were measured by ELISA for MPO. The basal level of MPO is represented by the mean level measured in serum samples of 30 healthy volunteers. The precent reduction in MPO out of the increase detected at C1D1 PRE-DOSE vs the basal level was calculated for each sample, and mean and significance were calculated for the 10 PDAC and 2 CRC patients (FIG. 4A), as well as for the 10 PDAC patients only (FIG. 4B). A significant reduction in MPO levels, indicating a decrease in NETs levels was observed in patient's serum following treatment with CM24 and Nivolumab.

Example 5: Clinical Trial Results Showing High Pretreatment Levels and a Significant Suppression of Serum MPO in Part C2 Patients Who Demonstrate Disease Control Following Treatment with CM24, Nivolumab and Chemotherapy

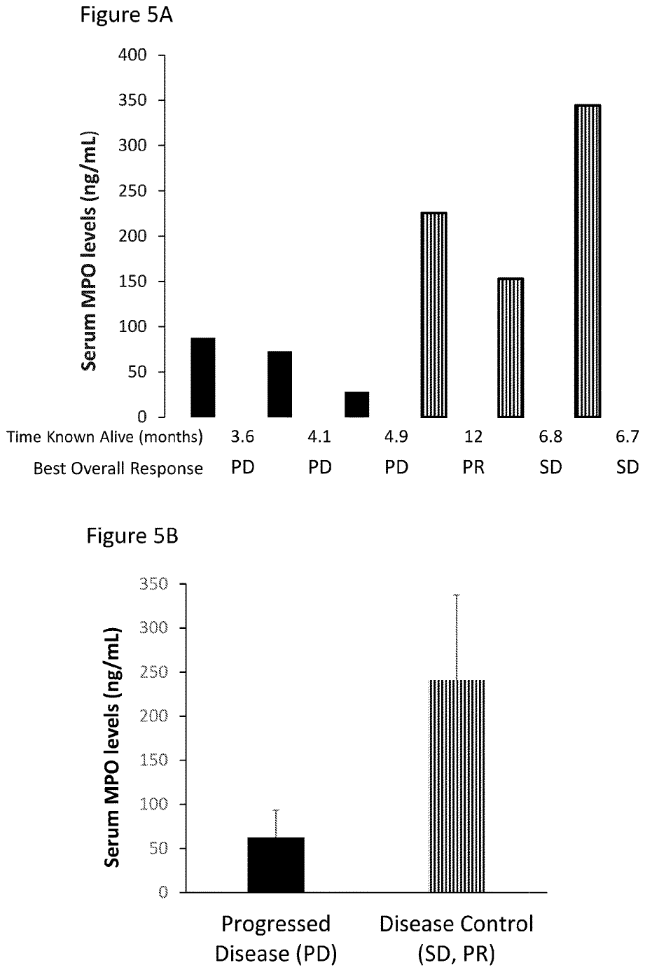

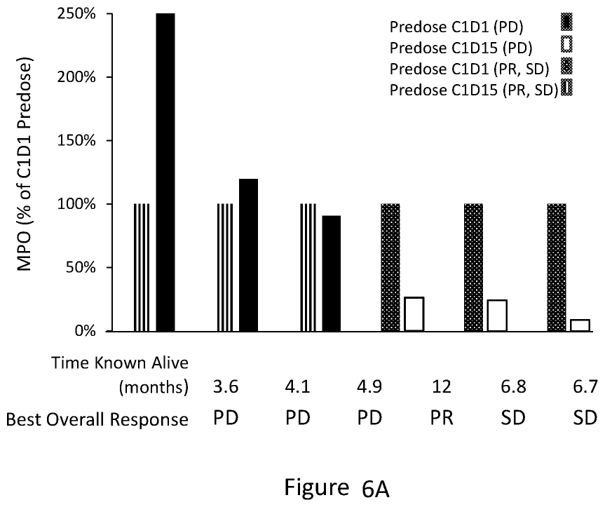

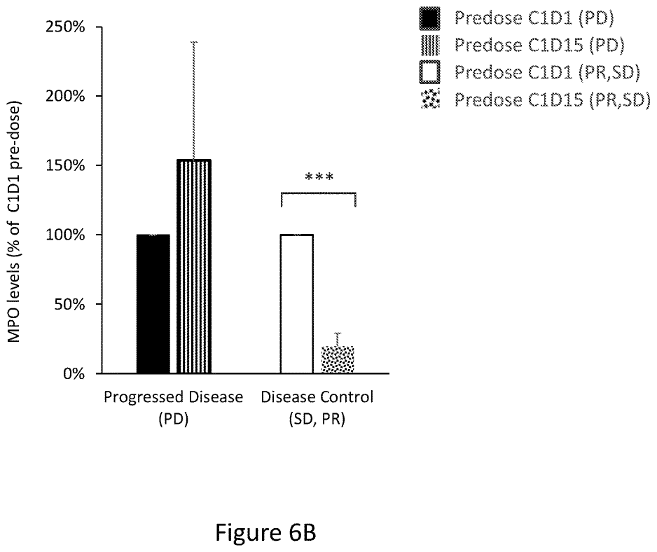

In part C2 of clinical trial NCT04731467, patients were administered with 20 mg/kg of CM24 followed by 30-minutes infusion of Nivolumab 480 mg/patient. The dosage of Nal-IRI was 70 mg/m2 intravenously over 90 minutes. Leucovorin (LV) at 200 400 mg/m2 was administered via a slow injection of at least 3 minutes at the end of Nal-IRI infusion subsequent to which, infusion of 2400 mg/m2 5FU was initiated. Serum samples were collected before and after treatment and tested for MPO levels by ELISA. In part C2 of clinical trial NCT04731467 six out of eight recruited patients, were treated more than twice, and therefore included in the analysis of serum MPO levels vs disease control. Results are presented in FIGS. 5 and 6. FIG. 5A shows the MPO levels in the pretreatment sera of patients. Patients who showed disease control following treatment including partial response (PR) or stable disease (SD), had significantly higher pre-dose serum MPO levels compared to patients demonstrating progressed disease (PD) or healthy volunteers. FIG. 5B depicts the average MPO levels in the pretreatment sera of patients who showed PR or SD (241 ng/mL) versus those who showed progressed disease (63 ng/mL), demonstrating a significant difference (p<0.05). FIG. 6 depicts the percentage of MPO reduction following treatment, in each patient (FIG. 6A) and as mean change (FIG. 6B), comparing patients who exhibited disease control (SD, PR) and those who progressed (PD). As can be seen in FIGS. 5A-5B, a significant difference (p<0.05) in pretreatment serum MPO levels between patients who demonstrate disease control and higher survival to patients who progressed was demonstrated, suggesting serum MPO as a potential biomarker for this treatment. FIGS. 6A-6B show a significant reduction in serum MPO following treatment, only in patients who showed disease control (P=0.0001) and not in patients who progressed on treatment.

CM24 interferes with CEACAM1-mediated NET activity, as demonstrated in-vitro, and led to a significant reduction in the levels of circulatory NETs in patients. The ability to unwrap the protective shield provided by NETs, which allow for immune evasion, metastasis, thrombosis, and other pathological processes, provides rationale for the use of CM24 to combat these life threatening conditions.

### Example 6: The Effect of CM24 on NET-Induced Platelet Aggregation

A platelet aggregation assay was developed using freshly drawn blood based on (Melissa V. Chan, Platelets; 2018; 29:7, 650-655). Platelet rich plasma (PRP) was incubated with prothrombotic agent adenosine diphosphate (ADP), in a 96 well flat bottom plate. The assay was read as absorbance at 595 nm with intermittent shaking and reading over 30 minutes, and aggregation of platelets was observed as a drop in absorbance. Untreated PRP and platelet poor plasma (PPP, plasma with platelets pelleted out) wells were included on every plate (and read at every time point) so that aggregation could be expressed as a percentage, using PRP and PPP as 0% and 100%, respectively. For NET stimulation of platelet aggregation. PRP was incubated with fresh and frozen NETs (collected from PMA-stimulated neutrophils).

Materials

| Item | Description |
|---|---|
| CM24 | Anti-CEACAM1 monoclonal antibody |
| Human IgG4 | Bio X Cell, #CP147, Lot#821321D1 |
| isotype control | |
| ADP | Cayman #16778, Batch #0473913-36 |
| Frozen NETs | Cayman #9004186, Batch#0669312, 0669313 |
| Fresh NETs | Generated in house |
| Calcein AM | Cayman #14948, Batch #0619818-25 |
| 96 well plate | Greiner #655101, Lot #E22113L7 |
| Plate reader | Biotek, Cytation5 imaging reader |
| Blood collection | 2.7 ml sodium citrate anti-coagulant BD plastic |
| tubes | blood collection tubes cat #363083, Lot |
| | #3016600 |
| Tyrodes-HEPES | Sodium chloride (VWR, #BDH9286) 134 mM |
| buffer | Potassium chloride (Chem-Impex, #01496) 2.9 mM |
| | Disodium phosphate (Chem-Impex #01755) 0.34 mM |
| | Sodium bicarbonate (MP, #194847) |
| | HEPES (Chem-Impex, #00174), 20 mM |
| | Magnesium chloride tetradydrate (Chem-Impex, |
| | #01496) 1 mM D(+) Glucose (Chem-Impex, #00805) |
| | 5 mM in water and pH adjusted to 7.4. |

### NET Isolation

Freshly drawn neutrophils and polymorphonucler cells (PMN) were stimulated for 4 hours with 100 nM Phorbol 12-myristate 13-acetate (PMA) and cell-free NETs were isolated, washed and stored in PBS at 4° C. for a maximum of 24 hours. Frozen NETs (frozen as cell-free NET pellets) were thawed and resuspended in PBS immediately prior to use in the assay.

Basic assay included isolation of platelet-rich plasma (PRP) and plasma from healthy donors. NET-induced aggregation was evaluated with fresh and frozen NETs. Using optimal NET induction conditions identified above, CM24 (500 μg/ml) and isotype control were added to the assay to assess the antibody effect on platelet aggregation.

### Platelet Rich/Poor Plasma (PRP/PPP) Preparation:

1. 40 ml of blood was collected.
   a. Note: The donor should not have taken aspirin, antihistamines, antibiotics, or anti-platelet drugs 14 days prior to blood draw.
2. Blood was pooled and centrifuged at 175×g for 20 minutes at 25° C. in a swinging bucket rotor with minimal brake.
3. The top yellow platelet rich plasma (PRP) was collected, and platelets were counted using 2 μg/ml of Calcein AM, incubating for 20 minutes at 37° C. and counted for green fluorescent cells on Nexcelom cell counter.
4. About 1 ml of the PRP was centrifuged at 15,000×g for 2 minutes. The supernatant, platelet poor plasma (PPP) was collected.

### Assay Set-Up:

1. Antibodies (hIgG4, and CM24) were added to PRP and incubated for 30 minutes at 37° C.
2. 5 μl of NETs were added to appropriate wells of a clear flat bottom 96 well plate. dilutions were made in Tyrodes-HEPES buffer.
3. 95 μl of PPP/PRP/PRP+Antibody was added and absorbance read at 595 nm
   a. Shaking between reads was either in the plate reader with 807 rpm at 37° C. or microplate reader at 1000 rpm at room temperature.

The percentage of aggregation was calculated using PRP only as 0% and PPP at 100% for every well at every time point using the following equation:

$$\% = 100 \times \frac{PRP - x}{PRP - PPP}$$

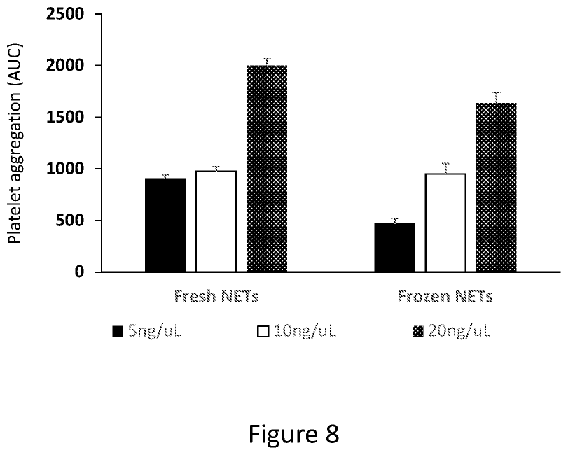

The results shown in FIG. 8 and Table 5 indicate that both fresh and frozen NETs at 5, 10 and 20 ng/μl, were able to efficiently induce aggregation of platelets, in a dose dependent manner.

TABLE 5
NET concentration-dependent platelet aggregation (AUC).

| | | Fresh | Frozen |
|---|---|---|---|
| 5 ng/μl | Mean | 907.7 | 468.5 |
| | SEM | 36.0 | 51.7 |
| | N | 4 | 4 |
| 10 ng/μl | Mean | 975.8 | 950.5 |
| | SEM | 47.9 | 101.9 |
| | N | 4 | 4 |
| 20 ng/μl | Mean | 1997.0 | 1636.0 |
| | SEM | 68.8 | 107.2 |
| | N | 4 | 4 |

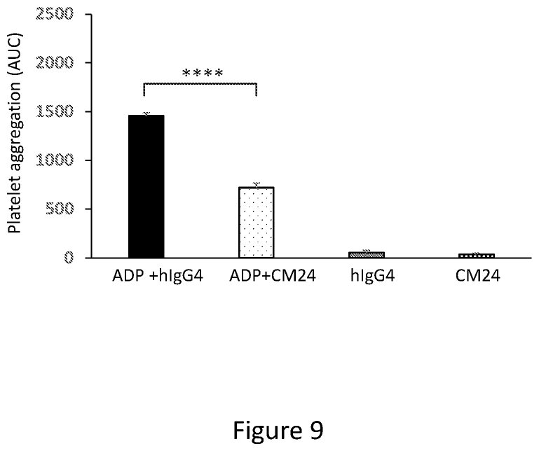

To evaluate the anti-thrombotic effect of CM24 in vitro, CM24 or its isotype control were added to the ADP-induced platelet aggregation assay described above. PRP was incubated with CM24 or hIgG4 at 500 μg/ml for 30 minutes at 37° C. Pretreated platelets were combined with ADP in the assay plate, and aggregation was quantified as described in the methods section. As shown in FIG. 9 and Table 6, CM24 significantly inhibited the ADP induced platelet aggregation as compared to isotype control.

TABLE 6
CM24 or isotype control in ADP-induced
platelet aggregation assay.

| | 30 min pretreat | | |
|---|---|---|---|
| | Mean | SEM | N |
| ADP + hIgG4 | 1456.0 | 32.5 | 3 |
| ADP + CM24 | 720.7 | 47.3 | 3 |
| hIgG4 | 53.1 | 26.7 | 3 |
| CM24 | 36.6 | 14.4 | 3 |

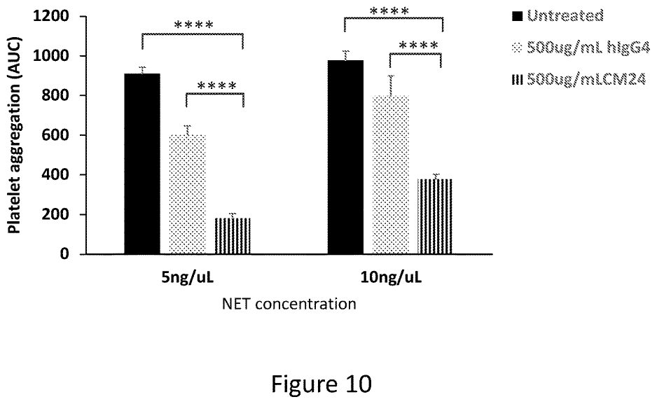

To evaluate the anti-thrombotic effect of CM24 in vitro, CM24 and its isotype control were added to the NET-induced platelet aggregation assay. PRP was incubated with CM24 or hIgG4 at 500 μg/ml for 30 minutes at 37° C. Pretreated platelets were combined with 5 and 10 ng/μl NETs in the assay plate, and aggregation was quantified as described in the methods section. As shown in FIG. 10 and Table 7, in the absence of treatment, NETs induced platelet aggregation. CM24 significantly inhibited the NET induced platelet aggregation as compared to non-treated and Isotype control.

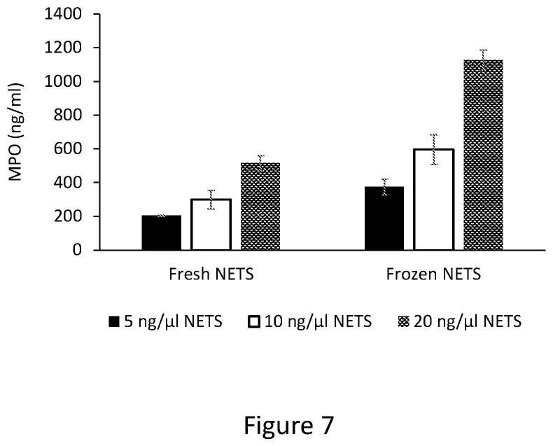

Summary data showing the percentage aggregation at assay endpoint are shown in Table 8 while FIG. 10 and Table 7 demonstrate the AUC of aggregation percentage over the course of the entire assay. At fresh NET concentrations of 5 and 10 ng/μl, addition of CM24 almost completely blocked NET-induced platelet aggregation. Isotype control antibody decreased platelet aggregation to a lesser degree, but the difference between CM24 and isotype control was highly significant when fresh NETs were used. The inhibition of platelet aggregation by CM24 exhibited complete blockade at 5 ng/μl NET, and significant inhibition at 10 ng/μl NET. The 5 ng/uL NETs is the most relevant concentration for use in vitro based on the levels of MPO measured in the patient's serum samples as exemplified in FIG. 7.

TABLE 7
Platelet aggregation with fresh NET induction. Inhibition
by CM24 is demonstrated vs. Isotype and untreated
controls. Data are mean AUC (% aggregation versus
time) values over the 30-minute experiment.

| | untreated | | | 500 μg/ml hIgG4 | | | 500 μg/ml CM24 | | |
|---|---|---|---|---|---|---|---|---|---|
| [NET] | Mean | SEM | N | Mean | SEM | N | Mean | SEM | N |
| 5 ng/μl | 907.7 | 36.0 | 4 | 599.5 | 28.5 | 4 | 179.2 | 26.5 | 4 |
| 10 ng/μl | 975.8 | 47.9 | 4 | 796.4 | 14.4 | 4 | 377.0 | 26.0 | 4 |

TABLE 8
Platelet aggregation with fresh NET induction and inhibitor treatments.
Data are percentage aggregation at the end of the study (30 min).

| | | untreated | | | 500 μg/ml hIgG4 | | | 500 μg/ml CM24 | | |
|---|---|---|---|---|---|---|---|---|---|---|
| NET | time | Mean | SEM | N | Mean | SEM | N | Mean | SEM | N |
| 5 ng/μl | 30 min | 37.7 | 2.2 | 4 | 12.6 | 2.5 | 4 | −5.3 | 1.9 | 4 |
| 10 ng/μl | 30 min | 37.3 | 7.7 | 4 | 28.9 | 1.8 | 4 | 11.5 | 3.2 | 4 |

Example 7: CM24 Interferes with Adhesion of CEACAM1 Expressing Cancer Cells to NETs.

Materials

| Material | Vendor | Item No. | Lot No. |
|---|---|---|---|
| CM24 | Provided by client | | |
| Human IgG4 isotype | Bio X Cell | CP147 | 821321D1 |
| control | | | |
| Dylight ™ 488 NHS | ThermoFisher | 46402 | YA3818991 |
| Ester | | | |
| CytoTell Red 650 | AAT Bioquest | 22255 | 255030 |
| Nuclear Blue ™ DCS1 | AAT Bioquest | 17548 | 107063 |
| NETs | Generated in-house | | |
| | with PMA | | |
| SK-MEL28 | In-house stock | | |

| Buffer | Composition |
|---|---|
| SK-MEL28 medium | EMEM (ATCC #30-2003) + 10% |
| | FBS |
| Jurkat medium | RPMI-1640 (ATCC #30-2001) + |
| | 10% FBS |
| Flow cytometry buffer | PBS + 1% BSA |

Fresh NETs were isolated and resuspended in PBS the day before the experiment and stored at 4° C. Frozen NETs were also used and resuspended in PBS. Both fresh and frozen NETs were stained on the day of the experiment.

Cancer cells were cultured according to ATCC recommendations. Cells were lifted using trypsin and stained with CytoTell Red (CTR) 650 in PBS and thoroughly washed to remove excess stain. Some cells were left unstained to use for compensation controls. After CTR staining, cells were incubated in a U-bottom polypropylene 96 well plate with 500 μg/ml CM24 or isotype control for 30 minutes at 37° C. Stained fresh and frozen NETs were then added to wells so that final concentration in each well was 4 ng/μl. NETs and cells were allowed to shake at 37° C. for 2 hours at 500 rpm. Cells were maintained in buffer in the presence of BSA, serum was absent. After incubation, cells were washed vigorously before being stained for Nuclear Blue in PBS+1% BSA, then acquired on the MASCQuant flow cytometer. Data were analyzed using FlowJo (TreeStar, Inc).

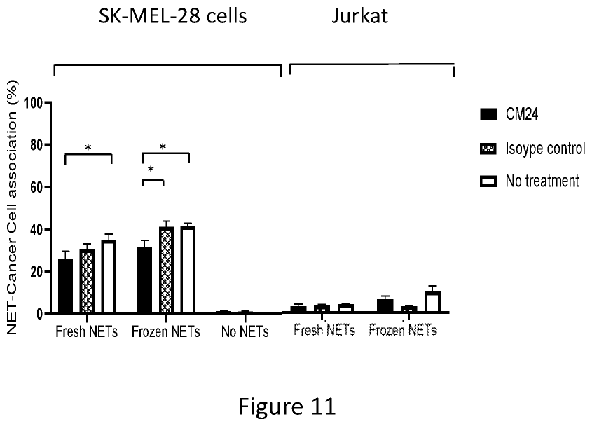

As detailed in Example 3 and Table 4 above, the adhesion of CEACAM1 positive human melanoma SK-MEL-28 cells to NETs was demonstrated by FACS analysis compared to adhesion to Jurtkat cells which do not express CEACAM1. Inhibition of this adhesion was further tested by utilizing CM24 or an isotype control. The results depicted in FIG. 11 demonstrate that CM24 significantly decreased the adhesion of SK-MEL-28 cancer cells to NETs while no significant effect of the isotype control was observed,

### Conclusions of the Experimental Results

NET induced platelet aggregation was demonstrated in a dose dependent manner using in vitro assays. CM24 significantly inhibited the NET induced platelet aggregation. At 5 ng/μl and 10 ng/μl fresh NETs, addition of CM24 reduced platelet aggregation by 70% and 53% respectively as compared to isotype control.

Platelet aggregation in vitro studies presented the potential of CM24 to inhibit both NET induced platelet aggregation as well as platelet aggregation induced by other stimuli such as ADP. These results suggest that blockade of CEACAM-1 using CM24 can effectively inhibit blood coagulation, suggesting the use of CM24 for inhibiting thrombosis and other non-malignant NET-associated conditions, as well as cancer associated thrombosis and metastasis.

Treatment of cancer cells with CM24 in vitro resulted with reduced binding of cancer cells to NETs and a decrease in the NET-induced cell migration. In the CM24 dose escalation part of a clinical trial study (NCT 04731467), advanced solid tumor patients (mainly PDAC and CRC patients) who received 2 prior lines of therapy were administered CM24 at 10, 15 and 20 mg/kg every other week and nivolumab at 480 mg every four weeks. In serum samples collected from the patients, the levels of MPO, a known NET marker, significantly decreased following treatment.

Expansion clinical phase of CM24 in combination with nivolumab and Nal-IRI/5FU/LV in patients with pancreatic adenocarcinoma was followed. In serum samples collected from these patients, high pre-treatment levels of serum MPO were detected in patients who later showed disease control (PR, SD), while patients who did not respond (PD) showed low pre-treatment levels of MPO similar to those observed in healthy donors. In addition, a significant decrease in MPO levels were observed in patients who showed disease control (PR, SD) to CM24, as opposed to those that did not (PD), which suggests CM24 as a potential treatment for NET related diseases and complications and for inhibition of cancer invasion and metastatic spread.

The above observations suggest that serum NET markers, for example MPO, may be used as a biomarker for selecting patients for anti CEACAM1 treatment and for monitoring and staging treated patients.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-58. (canceled)

59. A method of treating a non-malignant NET-related condition in a subject in need of such treatment, the method comprising:
(i) determining the level of at least one NET-biomarker in a biological sample obtained from the subject;
(ii) comparing the at least one NET-biomarker level to a reference value or to a control sample value; and
(iii) administering anti-CEACAM1 monoclonal antibody (mAb) or active fragment thereof to said subject if the at least one NET-biomarker level in the sample is significantly higher than the reference value or the control sample value.

60. The method of claim 59, wherein the NET-biomarker is myeloperoxidase (MPO).

61. The method of claim 60, wherein the significantly higher MPO level is equivalent to at least about 100%, at least about 200%, or at least about 300% increase relative to the reference value or to the control sample value.

62. The method of claim 59, wherein the non-malignant NET-related condition is a disease or disorder selected from thrombotic diseases, thrombosis, disease-associated thrombosis, pro-thrombosis condition, thromboinflammatory condition, venous thromboembolism, arterial thromboembolism, cardiovascular condition, pulmonary disease, hematological disease, autoimmune disease, autoinflammatory disease or disorder, immune-mediated disease, infectious disease, and systemic inflammatory condition.

63. The method of claim 62, wherein the thrombotic disease is a thrombotic cardiovascular disease.

64. The method of claim 62, wherein the hematological disease is thrombotic thrombocytopenia purpura (TTP), or heparin-induced thrombocytopenia or thrombosis.

65. The method of claim 59, wherein the anti-CEACAM1 mAb of active fragment, comprises a set of six CDR sequences wherein heavy chain CDR1 (HC-CDR1) comprises the sequence GYAFTNNLIE (SEQ ID NO: 1), heavy chain CDR2 (HC-CDR2) comprises the sequence VINPGSGDTNYNEKFKG (SEQ ID NO: 2), heavy chain CDR3 (HC-CDR3) comprises the sequence GDYYGGFAVDY (SEQ ID NO: 3), light chain CDR1 (LC-CDR1) comprises the sequence RTSQDIGNYLN (SEQ ID NO: 4), light chain CDR2 (LC-CDR2) comprises the sequence YTSRLHS (SEQ ID NO: 5), and light chain CDR3 (LC-CDR3) comprises the sequence QQGKSLPRT (SEQ ID NO: 6).

66. The method of claim 65, wherein the anti-CEACAM1 mAb is CM24 comprising a heavy chain variable region comprising the sequence set forth in SEQ ID NO: 7, and a light chain variable region comprising the sequence set forth in SEQ ID NO: 8, or an active fragment thereof, or an antibody analog or derivative thereof having at least 90% identity with any of said chain sequences.

67. A method of selecting a cancer patient amenable to a treatment with an anti-CEACAM1 mAb of active fragment thereof, comprising the steps of: (i) providing a biological sample from the subject; (ii) determining the level of MPO in the sample of step (i), and (iii) comparing the MPO level to a reference value or to a control sample value, wherein an increase in the level of MPO relative to the reference value or to the control sample value indicates that the subject is likely to respond therapeutically to the anti-CEACAM1 mAb or active fragment thereof.

68. The method of claim 67, wherein the significantly higher MPO level is equivalent to at least about 100%, at least about 200%, or at least about 300% increase relative to the reference value or to the control sample value.

69. The method of claim 67, wherein the biological sample is a blood sample or a biopsy.

70. The method of claim 67, wherein the anti-CEACAM1 mAb is CM24 comprising a heavy chain variable region comprising the sequence set forth in SEQ ID NO: 7, and a light chain variable region comprising the sequence set forth in SEQ ID NO: 8, or an active fragment thereof, or an antibody analog or derivative thereof having at least 90% identity with any of said chain sequences.

71. The method of claim 67, wherein treatment resulted in preventing, delaying or inhibiting treatment-induced thrombosis in the patient.

72. A method of preventing, delaying or inhibiting thrombosis in a subject, comprising administering to a subject in need thereof, an anti-CEACAM1 mAb, or an active fragment thereof.

73. The method of claim 72, wherein thrombosis is treatment-induced thrombosis, and wherein the treatment is selected from immunotherapy, surgery, hormone-therapy, and chemotherapy.

74. The method of claim 72, wherein the anti-CEACAM1 mAb is CM24 comprising a heavy chain variable region comprising the sequence set forth in SEQ ID NO: 7, and a light chain variable region comprising the sequence set forth in SEQ ID NO: 8, or an active fragment thereof, or an antibody analog or derivative thereof having at least 90% identity with any of said chain sequences.

75. A kit for predicting the response of a subject to an anti-CEACAM1 mAb treatment, the kit comprises means for determining the level of at least one NET-biomarker in a biological sample, means for comparing the expression level of the NET-biomarker to a reference value or to a control sample value; and instruction material directing the correlation between the ratio of the NET-biomarker to the reference or control levels.

76. The kit of claim 75, wherein the subject is diagnosed with a non-malignant NET-related disease, disorder, or complication.

77. The kit of claim 75, wherein the at least one NET-biomarker is MPO.

78. The kit of claim 75, wherein the anti-CEACAM1 mAb is CM24 comprising a heavy chain variable region comprising the sequence set forth in SEQ ID NO: 7, and a light chain variable region comprising the sequence set forth in SEQ ID NO: 8, or an active fragment thereof, or an antibody analog or derivative thereof having at least 90% identity with any of said chain sequences.
